(19) United States
(12) Patent Application Publication
(10) Pub. No.: US 2026/0327666 A1
(43) Pub. Date: Oct. 22, 2026

(54) METHOD FOR SYNTHESIZING GLABRIDIN

(71) Applicant: TSINGHUA UNIVERSITY, BEIJING (CN)

(72) Inventors: CHUN LI, BEIJING (CN); ZHEN ZHANG, BEIJING (CN); LEI QIN, BEIJING (CN)

(21) Appl. No.: 19/561,545

(22) Filed: Mar. 10, 2026

(30) Foreign Application Priority Data
Mar. 11, 2025 (CN) 2025102805925

(51) Int. Cl. C12P 17/18; C12N 1/18; C12N 9/02; C12N 9/04; C12N 9/10; C12N 15/81; C12R 1/84; C12R 1/865

(52) U.S. Cl. CPC C12P 17/181; C12N 1/18; C12N 9/0006; C12N 9/001; C12N 9/0073; C12N 9/1007; C12N 9/1085; C12N 15/815; C12R 2001/84; C12R 2001/865; C12Y 101/01; C12Y 101/01246; C12Y 103/01045; C12Y 114/13081; C12Y 114/14; C12Y 201/01; C12Y 205/01

(57) ABSTRACT

Provided is a series of enzyme for synthesizing glabridin, a method for biosynthesizing glabridin, and an engineered biological host. The series of enzymes for synthesizing glabridin derived from Glycyrrhiza glabra, including a 4′-O-methyltransferase, an isoflavone 2′-hydroxylase, an isoflavone reductase, a vestitone reductase, a prenyltransferase, a pterocarpan reductase, an oxidative cyclase, and a demethylase, which are first disclosed, exhibit respective activities in various biological hosts.

### CROSS-REFERENCE TO RELATED PATENT APPLICATION

This application claims priority to and the benefit of the Chinese patent application No. 2025102805925 filed on Mar. 11, 2025, the entire content of which is incorporated herein by reference.

### STATEMENT REGARDING SEQUENCE LISTING

This application contains a sequence listing in XML format, which has been submitted electronically and is hereby incorporated by reference in its entirety. The sequence listing, created on May 18, 2026, is named 115P000374US-SEQUENCELISTING.xml and is 69,845 bytes in size.

### FIELD OF THE DISCLOSURE

The present disclosure relates to the technical field of bioengineering and technology, in particular to an enzyme for synthesizing glabridin, a method for biosynthesizing glabridin, and an engineered biological host.

### BACKGROUND OF THE DISCLOSURE

Glabridin is a flavonoid compound extracted from Glycyrrhiza glabra L, having various physiological activities, such as anti-inflammatory property, antioxidant property, antibacterial property, tyrosinase inhibitory activity and the like. Therefore, glabridin is widely used in the field of brightening and skincare, for example, often as an ingredient in various cosmetics. In addition, glabridin has shown anti-cancer activity in a certain extent as well as potential values of application in aspects of anti-allergy, anti-virus, and cardiovascular system protection. Due to the natural origin and multiple pharmacological properties of the glabridin, it has gradually become a research hotspot in the fields of drug development, medical aesthetics, and functional foods.

At present, the glabridin is extracted mainly by the separation and purification from the roots of the plant Glycyrrhiza glabra (Glycyrrhiza glabra L.). This extraction method, however, is fraught with limitations stemming from multiple factors. For one, the glabridin content in Glycyrrhiza glabra is low, accounting for only approximately 0.09% of the plant's composition. What's more, the extraction process entails multiple steps including drying, grinding and comminution, often coupled with auxiliary extraction techniques such as ultrasonic and microwave treatment, which result in a cumbersome workflow, low extraction efficiency, high costs and significant environmental pollution. In addition, Glycyrrhiza features a well-developed root system with strong windbreak and sand-fixation capabilities. It is mainly distributed in Central Asia and northwest China, and in China, distributed in western regions such as Gansu, Xinjiang and Inner Mongolia with desert and semi-desert regions, endowing it with an ecological value. However, the above-mentioned uprooting extraction method purposes severely damages Glycyrrhiza vegetation and the local ecological environment. The Glycyrrhiza used at present is mostly derived from Glycyrrhiza imported and artificially cultivated. However, artificially cultivated Glycyrrhiza has drawbacks like a long growth cycle, occupation of arable land and high susceptibility to climatic conditions, all of which severely constrain glabridin production.

Therefore, there is an urgent need for an efficient and environmentally friendly method for synthesizing glabridin in the related art.

### SUMMARY OF THE DISCLOSURE

The present disclosure aims to solve at least one of the technical problems in the related art to a certain extent.

Therefore, in a first aspect, embodiments of the present disclosure provide a method for biosynthesizing glabridin, including: providing a biological host; engineering the biological host to heterologously express enzymes of a 4′-O-methyltransferase, an isoflavone 2′-hydroxylase, an isoflavone reductase, a vestitone reductase, a prenyltransferase, a pterocarpan reductase, an oxidative cyclase, and a demethylase to obtain an engineered biological host; and culturing the engineered biological host to obtain the glabridin, wherein the enzymes heterologously expressed in the engineered biological host consist of the 4′-O-methyltransferase with an amino acid sequence of SEQ ID NO: 1 or SEQ ID NO: 2, the isoflavone 2′-hydroxylase with an amino acid sequence of SEQ ID NO: 3 or SEQ ID NO: 4, the isoflavone reductase with an amino acid sequence of SEQ ID NO: 5 or SEQ ID NO: 6, the vestitone reductase with an amino acid sequence of SEQ ID NO: 7 or SEQ ID NO: 8, the prenyltransferase with an amino acid sequence of SEQ ID NO: 16 or SEQ ID NO: 10, the pterocarpan reductase with an amino acid sequence of SEQ ID NO: 11 or SEQ ID NO: 12, the oxidative cyclase with an amino acid sequence of SEQ ID NO: 13, and the demethylase with an amino acid sequence of SEQ ID NO: 14 or SEQ ID NO: 15, wherein the biological host is yeast, wherein the glabridin is biosynthesized using daidzein as a substrate.

In some embodiments, wherein the engineered biological host heterologously expresses the 4′-O-methyltransferase with an amino acid sequence of SEQ ID NO: 1, the isoflavone 2′-hydroxylase with an amino acid sequence of SEQ ID NO: 3, the isoflavone reductase with an amino acid sequence of SEQ ID NO: 5, the vestitone reductase with an amino acid sequence of SEQ ID NO: 7, the prenyltransferase with an amino acid sequence of SEQ ID NO: 16, the pterocarpan reductase with an amino acid sequence of SEQ ID NO: 11, the oxidative cyclase with an amino acid sequence of SEQ ID NO: 13, and the demethylase with an amino acid sequence of SEQ ID NO: 14.

In some embodiments, wherein the engineered biological host heterologously expresses the 4′-O-methyltransferase with an amino acid sequence of SEQ ID NO: 2, the isoflavone 2′-hydroxylase an the amino acid sequence of SEQ ID NO: 4, the isoflavone reductase with an amino acid sequence of SEQ ID NO: 6, the vestitone reductase with an amino acid sequence of SEQ ID NO: 8, the prenyltransferase with an amino acid sequence of SEQ ID NO: 10, the pterocarpan reductase with an amino acid sequence of SEQ ID NO: 12, the oxidative cyclase with an amino acid sequence of SEQ ID NO: 13, and the demethylase with an amino acid sequence of SEQ ID NO: 15.

In some embodiments, wherein the yeast includes Saccharomyces cerevisiae, Yarrowia lipolytica, and Pichia pastoris.

Compared with the related art, the embodiments of the present disclosure achieve at least the following beneficial effects:

The present disclosure identifies a series of enzymes associated with glabridin biosynthesis from the plant Glycyrrhiza glabra, and enables the de novo biosynthesis of glabridin by heterologously expressing the identified enzymes in vitro, which include O-methyltransferase, isoflavone 2′-hydroxylase, isoflavone reductase, vestitone reductase, prenyltransferase, pterocarpan reductase, oxidative cyclase, and demethylase. Particularly, with use of the highly efficient and specific methyltransferases, prenyltransferases, pterocarpan reductases, oxidative cyclases, and demethylases screened, the present disclosure realizes, for the first time, the heterologous biosynthesis of glabridin from daidzein as raw material via biocatalysis, with the glabridin yield reaching over 60%.

The complete pathway and method for biosynthesizing glabridin according to the embodiments of the present disclosure are simple, efficient, and environmentally friendly, making it a promising approach for the large-scale industrial production of glabridin. This approach is expected to replace the existing plant extraction method for glabridin production, which relies on uprooting the plant and suffers from long production cycles, low efficiency, and prolonged occupation of arable land.

### DETAILED DESCRIPTION OF THE EXEMPLARY EMBODIMENTS

The embodiments of the present disclosure are described below in combination with the detailed description in detail, and the embodiments given are illustrative only, not limiting the scope of the present disclosure. The embodiments provided below may serve as a guide for those skilled in the art to make further improvements, but do not constitute a limitation of the present disclosure in any way.

The present disclosure is made based on the following understanding of the inventors:

The related method, for extracting glabridin from the plant by uprooting, is inherently unsustainable and runs counter to the development of a green, eco-friendly ecological environment, for its long production cycles, low efficiency and prolonged occupation of arable land. Therefore, it is necessary to develop an innovative method that alter raw material sources to address these issues. Chemical synthesis methods, on the other hand, suffer from drawbacks including low yields, the requirement for product chiral resolution, harsh reaction conditions and complex operations. In the related art, research for the biosynthesis of glabridin has been limited to its precursor compounds such as 4′-O-methylpreglabridin, medicarpin and the like.

The inventors of the present disclosure identify a series of enzymes associated with glabridin biosynthesis from the plant Glycyrrhiza glabra. The present disclosure not only achieves the in-vitro biosynthesis of glabridin for the first time, but also enables the biosynthesis of glabridin using daidzein as a substrate, by expressing the identified enzymes in vitro, which include O-methyltransferase, isoflavone 2′-hydroxylase, isoflavone reductase, vestitone reductase, prenyltransferase, pterocarpan reductase, oxidative cyclase, and demethylase. Particularly, with use of the highly efficient and specific methyltransferases, prenyltransferases, pterocarpan reductases, oxidative cyclases, and demethylases screened, the present disclosure realizes, for the first time, the heterologous biosynthesis of glabridin from daidzein as raw material via biocatalysis, with the glabridin yield reaching over 60%.

The complete pathway and method for biosynthesizing glabridin according to the embodiments of the present disclosure are simple, efficient, and environmentally friendly, making it a promising approach for the large-scale industrial production of glabridin. This approach is expected to replace the existing plant extraction method for glabridin production, which relies on uprooting the plant and suffers from long production cycles, low efficiency, and prolonged occupation of arable land.

In a first aspect, embodiments of the present disclosure provide an enzyme for synthesizing glabridin, the enzyme includes one or more enzymes selected from: a 4′-O-methyltransferase with an amino acid sequence depicted as SEQ ID NO: 2; an isoflavone 2′-hydroxylase with an amino acid sequence depicted as SEQ ID NO: 4; an isoflavone reductase with an amino acid sequence depicted as SEQ ID NO: 6; a vestitone reductase with an amino acid sequence depicted as SEQ ID NO: 8; a prenyltransferase with an amino acid sequence depicted as SEQ ID NO: 10; a pterocarpan reductase with an amino acid sequence depicted as SEQ ID NO: 11 or 12; an oxidative cyclase with an amino acid sequence depicted as SEQ ID NO: 13; a demethylase with an amino acid sequence depicted as SEQ ID NO: 15.

In a second aspect, embodiments of the present disclosure provide a method for biosynthesizing glabridin, including: providing a biological host; enabling the biological host to express one or more enzymes selected from the group consisting of a 4′-O-methyltransferase, an isoflavone 2′-hydroxylase, an isoflavone reductase, a vestitone reductase, a prenyltransferase, a pterocarpan reductase, an oxidative cyclase, and a demethylase to obtain an engineered biological host; and culturing the engineered biological host to obtain the glabridin.

In some embodiments, wherein an amino acid sequence of the 4′-O-methyltransferase is selected from one or more of SEQ ID NO: 1 and SEQ ID NO: 2.

In some embodiments, an amino acid sequence of the isoflavone 2′-hydroxylase is selected from one or more of SEQ ID NO: 3 and SEQ ID NO: 4.

In some embodiments, an amino acid sequence of the isoflavone reductase is selected from one or more of SEQ ID NO: 5 and SEQ ID NO: 6.

In some embodiments, an amino acid sequence of the vestitone reductase is selected from one or more of SEQ ID NO: 7 and SEQ ID NO: 8.

In some embodiments, an amino acid sequence of the prenyltransferase is selected from one or more of SEQ ID NO: 9, SEQ ID NO: 10, and SEQ ID NO: 16.

In some embodiments, an amino acid sequence of the pterocarpan reductase is selected from one or more of SEQ ID NO: 11 and SEQ ID NO: 12.

In some embodiments, an amino acid sequence of the oxidative cyclase is selected from SEQ ID NO: 13.

In some embodiments, an amino acid sequence of the demethylase is selected from one or more of SEQ ID NO: 14 and SEQ ID NO: 15.

In some embodiments, wherein an amino acid sequence of the 4′-O-methyltransferase is depicted as SEQ ID NO: 2.

In some embodiments, an amino acid sequence of the isoflavone 2′-hydroxylase is depicted as SEQ ID NO: 4.

In some embodiments, an amino acid sequence of the isoflavone reductase is depicted as SEQ ID NO: 6.

In some embodiments, an amino acid sequence of the vestitone reductase is depicted as SEQ ID NO: 8.

In some embodiments, an amino acid sequence of the prenyltransferase is depicted as SEQ ID NO: 10.

In some embodiments, an amino acid sequence of the pterocarpan reductase is depicted as SEQ ID NO: 11 or SEQ ID NO: 12.

In some embodiments, an amino acid sequence of the oxidative cyclase is depicted as SEQ ID NO: 13.

In some embodiments, an amino acid sequence of the demethylase is depicted as SEQ ID NO: 15.

In some embodiments, wherein the biological host is selected from the group consisting of a gram-positive bacterium, a gram-negative bacterium, an actinomycete, a yeast, a filamentous fungus, and a Nicotiana species.

In some embodiments, the gram-positive bacterium includes Bacillus subtilis and Corynebacterium glutamicum, the gram-negative bacterium includes Escherichia coli, the actinomycete includes Streptomyces coelicolour, the yeast includes Saccharomyces cerevisiae, Yarrowia lipolytica, and Pichia pastoris, the filamentous fungus includes Aspergillus niger, and the Nicotiana species comprises Nicotiana benthamiana.

In some embodiments, wherein the glabridin is biosynthesized using a substrate selected from the group consisting of daidzein, formononetin, vestitone, and medicarpin.

In some embodiments, wherein the biological host is Nicotiana benthamiana, wherein the engineered biological host heterologously expresses the prenyltransferase with an amino acid sequence depicted as SEQ ID NO: 10, the pterocarpan reductase with an amino acid sequence depicted as SEQ ID NO: 11, the oxidative cyclase with an amino acid sequence depicted as SEQ ID NO: 13, and the demethylase with an amino acid sequence depicted as SEQ ID NO: 15, and wherein a substrate for biosynthesizing the glabridin is medicarpin.

In some embodiments, wherein the biological host is yeast, wherein the engineered biological host heterologously expresses the 4′-O-methyltransferase with an amino acid sequence shown in SEQ ID NO: 1, the isoflavone 2′-hydroxylase with an amino acid sequence shown in SEQ ID NO: 3, the isoflavone reductase with an amino acid sequence depicted as SEQ ID NO: 5, the vestitone reductase with an amino acid sequence depicted as SEQ ID NO: 7, the prenyltransferase with an amino acid sequence depicted as SEQ ID NO: 16, the pterocarpan reductase with an amino acid sequence depicted as SEQ ID NO: 11, the oxidative cyclase with an amino acid sequence depicted as SEQ ID NO: 13, and the demethylase with an amino acid sequence depicted as SEQ ID NO: 14, and wherein a substrate for biosynthesizing the glabridin is daidzein.

In some embodiments, wherein the biological host is yeast, wherein the engineered biological host heterologously expresses the 4′-O-methyltransferase with an amino acid sequence depicted as SEQ ID NO: 2, the isoflavone 2′-hydroxylase an the amino acid sequence depicted as SEQ ID NO: 4, the isoflavone reductase with an amino acid sequence depicted as SEQ ID NO: 6, the vestitone reductase with an amino acid sequence depicted as SEQ ID NO: 8, the prenyltransferase with an amino acid sequence depicted as SEQ ID NO: 10, the pterocarpan reductase with an amino acid sequence depicted as SEQ ID NO: 12, the oxidative cyclase with an amino acid sequence depicted as SEQ ID NO: 13, and the demethylase with an amino acid sequence depicted as SEQ ID NO: 15, and wherein a substrate for biosynthesizing the glabridin is daidzein.

In a third aspect, embodiments of the present disclosure provide an engineered biological host, expressing one or more enzymes selected from the group consisting of: a 4′-O-methyltransferase with an amino acid sequence depicted as SEQ ID NO: 2; an isoflavone 2′-hydroxylase with an amino acid sequence depicted as SEQ ID NO: 4; an isoflavone reductase with an amino acid sequence depicted as SEQ ID NO: 6; a vestitone reductase with an amino acid sequence depicted as SEQ ID NO: 8; a prenyltransferase with an amino acid sequence depicted as SEQ ID NO: 10; a pterocarpan reductase with an amino acid sequence depicted as SEQ ID NO: 11 or SEQ ID NO: 12; an oxidative cyclase with an amino acid sequence depicted as SEQ ID NO: 13; a demethylase with an amino acid sequence depicted as SEQ ID NO: 15.

In a fourth aspect, embodiments of the present disclosure provide use of a 4′-O-methyltransferase with an amino acid sequence depicted as SEQ ID NO: 1 in synthesizing formononetin or glabridin.

In a fifth aspect, embodiments of the present disclosure provide use of an isoflavone 2′-hydroxylase with an amino acid sequence depicted as SEQ ID NO: 3 in synthesizing 2′-hydroxyformononetin or glabridin.

In a sixth aspect, embodiments of the present disclosure provide use of an isoflavone reductase with an amino acid sequence depicted as SEQ ID NO: 5 in synthesizing vestitone or glabridin.

In a seventh aspect, embodiments of the present disclosure provide use of a vestitone reductase with an amino acid sequence depicted as SEQ ID NO: 7 in synthesizing medicarpin or glabridin.

In an eighth aspect, embodiments of the present disclosure provide use of a prenyltransferase with an amino acid sequence depicted as SEQ ID NO: 9 or SEQ ID NO: 16 in synthesizing glabridin.

In a nineth aspect, embodiments of the present disclosure provide use of a demethylase with an amino acid sequence depicted as SEQ ID NO: 14 in synthesizing glabridin.

It can be understood that the uses according to the embodiments of the fourth to ninth aspects of the present disclosure include, but are not limited to, intracellular catalysis, extracellular catalysis, enzymatic catalysis, or cell-free catalysis.

The biological hosts applicable to the embodiments of the second aspect are identical to those for the uses set forth in the fourth to ninth aspects of the present disclosure, and will not be repeated here.

### Terms

As used herein, the terms “engineered strain”, “engineered plant”, and “engineered biological host” refer to a bacterial or fungal cell line or a plant that efficiently expresses exogenous genes via genetic engineering techniques.

As used herein, the terms “starting strain”, “chassis strain”, and “biological host” refer to an original strain of bacterium or fungus or an original plant used for genetic engineering.

As used herein, the term “genetic circuit” refers to a gene expression system containing all essential elements required for the expression of a target polypeptide, typically including the following elements: a promoter, an encoding reporter gene, and a terminator.

As used herein, the term “comprising” or “including” is an open-ended expression. When the term is used to describe a sequence of a protein or a nucleic acid, the protein or the nucleic acid may consist of the recited sequence alone, or may have additional amino acid or nucleotide residues at one or both ends, while still retaining the same or similar biological activity as the original sequence.

As used herein, the term “comprising” or “including” is an open-ended description that encompasses the specified components or steps, as well as other components or steps that do not substantially influence the disclosed technical solution.

As used herein, the term “and/or” includes all possible combinations of the items it connects, with each combination deemed to be individually recited herein. For example, “A and/or B” includes “A”, “A and B”, and “B”. In another example, “A, B and/or C” includes “A”, “B”, “C”, “A and B”, “A and C”, “B and C”, and “A, B and C”.

The abbreviations used herein and their full names are provided in Table 1 below.

TABLE 1

| Abbreviation | Full Name |
|---|---|
| GgOMT | 4′-O-methyltransferase derived from Glycyrrhiza glabra |
| PlOMT9 | 4′-O-methyltransferase derived from Pueraria lobata |
| MES | 2-(N-morpholino)ethanesulfonic acid |
| AS | Acetosyringone |
| GgI2′H | Isoflavone 2′-hydroxylase derived from Glycyrrhiza |
| | glabra |
| GeCYP81E1 | Isoflavone 2′-hydroxylase derived from Glycyrrhiza |
| | echinata |
| IPTG | Isopropyl-β-D-thiogalactopyranoside |
| GgIFR | Isoflavone reductase derived from Glycyrrhiza glabra |
| MsIFR | Isoflavone reductase derived from Medicago sativa |
| GgVR | Vestitone reductase derived from Glycyrrhiza glabra |
| MsVR | Vestitone reductase derived from Medicago sativa |
| GgPT | Prenyltransferase derived from Glycyrrhiza glabra |
| PcM4DT/tPcM4DT | pterocarpan prenyltransferase derived from Cullen |
| | corylifolium |
| GgPTR | Pterocarpan reductase derived from Glycyrrhiza glabra |
| GgOC | Oxidative cyclase derived from Glycyrrhiza glabra |
| GgDMT | Demethylase derived from Glycyrrhiza glabra |
| NhDMT | Demethylase derived from Nectria haematococca |

### Sources of Experimental Materials

Wild-type (WT) Nicotiana benthamiana is described in the article “Yuan J, Yu Z, Li Y, Shah SHA, Xiao D, Hou X, Li Y. Ectopic expression of BrIQD35 promotes drought stress tolerance in Nicotiana benthamiana. Plant Biol (Stuttg). 2022 August; 24(5):887-896.” The material is available to the public from the applicant for the purpose of repeating the experiments of the present disclosure only.

Streptomyces coelicolor (S. coelicolor) A3(2) is described in the article “Hopwood DA (1999) Forty years of genetics with Streptomyces: from in vivo through in vitro to in silico. Microbiology 145(9):2183-2202.” The material is available to the public from the applicant for the purpose of repeating the experiments of the present disclosure only.

Yarrowia lipolytica ATCC 201249 is described in the article “Du H-X, Xiao W-H, Wang Y, Zhou X, Zhang Y, Liu D, et al. (2016) Engineering Yarrowia lipolytica for Campesterol Overproduction. PLoS ONE 11(1): e0146773.” The material is available to the public from the applicant for the purpose of repeating the experiments of the present disclosure only.

The present disclosure is further illustrated with the specific examples of glabridin heterologous biosynthesis below. The specific examples are provided only for explanatory purposes and are not intended to limit the scope of the present disclosure. Without departing from the scope of the appended claims, those skilled in the art may make various modifications to the aspects of the present disclosure, and all such modifications shall also fall within the protection scope of the present disclosure. For example, those skilled in the art may understand and carry out the replacement of the cellular expression systems and vector plasmids used in the present examples with other conventional expression systems and vectors in the art.

The materials, reagents, etc. used in the following examples are commercially available unless otherwise specified.

### Example 1: Expression and Functional Verification of 4′-O-Methyltransferase

4′-O-methyltransferase derived from Glycyrrhiza glabra (GgOMT), with the amino acid sequence of SEQ ID NO: 2 and the nucleotide sequence of SEQ ID NO: 17 accordingly, was selected and subjected to gene synthesis according to the sequence provided. The synthesized gene was amplified by PCR and ligated into the Agrobacterium expression vector pBI121 using Gibson assembly to obtain a recombinant plasmid. The recombinant plasmid was introduced into Agrobacterium competent cells EHA105 by electroporation. Agrobacterium clones transformed with the expression plasmid successfully were picked and cultured in 1 ml of LB medium containing the corresponding antibiotics at 28° C. with shaking at 250 rpm for 24 hours. Then, 50 μl of cultured Agrobacterium solution was inoculated into 5 ml of LB medium containing the corresponding antibiotics as well as with 100 μl of 0.5 M MES and 2 μl of 100 mM AS added, and then further cultured at 28° C. with shaking at 250 rpm until OD600=1.0. A centrifugation at 4000 rpm for 10 minutes was performed, and the precipitate of bacterial cells was collected and resuspended in 10 mM MgCl₂ to OD600=1.0. Then, 2 μl of 100 mM AS was added per ml of bacterial resuspended solution, followed by standing for more than 3 hours.

Nicotiana benthamiana in the vigorous growth period was selected. The bacterial solution was drawn up into a syringe without a needle. With fingers pressing against the front of the leaf, the bacterial solution was infiltrated from the back of the leaf using the syringe. 1 mM daidzein solution as the reaction substrate was injected at the same time in the same way. The treated plant was kept for 24-48 hours, and then subjected to sample collection for product detection.

The collected leaves were cut into pieces and subjected to bead-beating disruption at −20° C. After the disruption, the precipitate and the separated supernatant were extracted with an equal volume of ethyl acetate, and the obtained organic phase was evaporated to dryness and redissolved with methanol. The production of formononetin was detected by ultra-performance liquid chromatography (UPLC) or liquid chromatography-mass spectrometry (LC-MS).

Specifically, quantitative detection of the fermentation products from the engineered strains was performed using a UPLC instrument (Agilent 1260). Separation was carried out on a Poroshell 120 EC-C18 column of particle size 2.7 μm and 3.0×100 mm. The detection wavelength was set at 254 nm, and the column oven temperature was maintained at 35° C. Mobile phase A consisted of 0.1% formic acid in water, and mobile phase B was acetonitrile. Gradient elution was performed as follows: 0 min, 20% B; 0-4.5 min, 20%-35% B; 4.5-6 min, 35%-50% B; 6-12 min, 50%-65% B; 12-18 min, 65%-85% B; 18-22.5 min, 85%-95% B; 22.5-24 min, 95%-20% B; and 24-30 min, 20% B. The flow rate was 0.2 mL/min, and the injection volume was 1 μL. Qualitative and quantitative detection were performed using the external standard method. The detection results confirmed the production of formononetin.

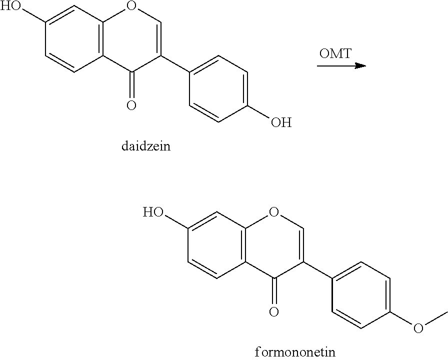

### Example 2: Expression and Functional Verification of Isoflavone 2′-Hydroxylase

Isoflavone 2′-hydroxylase derived from Glycyrrhiza glabra (GgI2′H), with the amino acid sequence of SEQ ID NO: 4 and the nucleotide sequence of SEQ ID NO: 18 accordingly, was selected and subjected to gene synthesis according to the sequence provided. The synthesized gene was amplified by PCR and ligated into the Escherichia coli expression vector pET28a using Gibson assembly to obtain a recombinant plasmid. The recombinant plasmid was introduced into Escherichia coli competent cells BL21 by electroporation. Escherichia coli clones transformed with the expression plasmid successfully were picked and cultured in 1 mL of LB medium containing the corresponding antibiotics at 37° C. with shaking at 220 rpm for 12 hours. Then, 0.1 mL of cultured bacterial solution was inoculated into 5 mL of LB medium containing the corresponding antibiotics, and cultured until the OD600=0.6-0.8 as determined using a UV spectrophotometer. The temperature of the shaker was reduced to 16° C., and 1 mM IPTG was added to the bacterial solution for induction. After 10 hours, 1 mM formononetin was added as the reaction substrate, the temperature of the shaker was adjusted to 30° C., and samples were collected for product detection after 24-48 hours of reaction.

The reacted culture solution was centrifuged, and the supernatant was collected and extracted with an equal volume of ethyl acetate. The obtained organic phase was evaporated to dryness and redissolved with methanol. The production of 2′-hydroxyformononetin was detected by UPLC or LC-MS.

Specifically, quantitative detection of the fermentation products from the engineered strains was performed using a UPLC instrument (Agilent 1260). Separation was carried out on a Poroshell 120 EC-C18 column of particle size 2.7 μm and 3.0×100 mm. The detection wavelength was set at 254 nm, and the column oven temperature was maintained at 35° C. Mobile phase A was 0.1% formic acid in water, and mobile phase B was acetonitrile. Gradient elution was performed as follows: 0 min, 20% B; 0-4.5 min, 20%-35% B; 4.5-6 min, 35%-50% B; 6-12 min, 50%-65% B; 12-18 min, 65%-85% B; 18-22.5 min, 85%-95% B; 22.5-24 min, 95%-20% B; and 24-30 min, 20% B. The flow rate was 0.2 mL/min, and the injection volume was 1 μL. Qualitative and quantitative detection were performed using the external standard method. The detection results confirmed the production of 0.96 mM 2′-hydroxyformononetin.

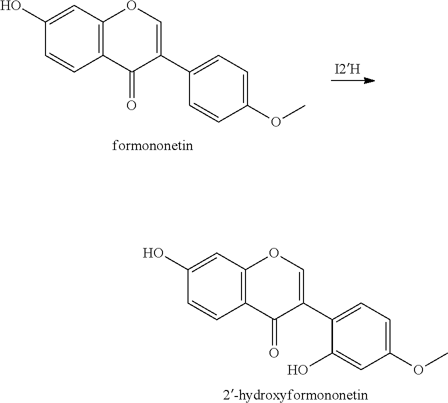

### Example 3: Expression and Functional Verification of Isoflavone Reductase

Isoflavone reductase derived from Glycyrrhiza glabra (GgIFR), with the amino acid sequence of SEQ ID NO: 6 and the nucleotide sequence of SEQ ID NO: 19 accordingly, was selected and subjected to gene synthesis according to the sequence provided. The synthesized gene was amplified by PCR and ligated into the Saccharomyces cerevisiae expression vector pYES2 using Gibson assembly to obtain a recombinant plasmid. The recombinant plasmid was introduced into Escherichia coli competent cells DH5a by electroporation. Escherichia coli clones transformed with the expression plasmid successfully were picked and cultured in 1 mL of LB medium containing the corresponding antibiotics at 37° C. with shaking at 220 rpm for 12 hours. The bacterial cells were collected by centrifugation to extract the plasmid. The plasmid was transformed into Saccharomyces cerevisiae INVSC cells. The positive clones obtained after transformation were inoculated into SD-Ura medium and cultured at 30° C. with shaking at 250 rpm for 16 hours. Then, 20 g/L galactose was added for induction, and 1 mM 2′-hydroxyformononetin was added as the reaction substrate at the same time. The reaction was carried out at 30° C. with shaking at 220 rpm for 24-48 hours, and samples were collected for product detection.

The reacted culture solution was centrifuged, and the supernatant was collected and extracted with an equal volume of ethyl acetate. The obtained organic phase was evaporated to dryness and redissolved with methanol. The production of vestitone was detected by UPLC or LC-MS.

Specifically, quantitative detection of the fermentation products from the engineered strains was performed using a UPLC instrument (Agilent 1260). Separation was carried out on a Poroshell 120 EC-C18 column of particle size 2.7 μm and 3.0×100 mm. The detection wavelength was set at 254 nm, and the column oven temperature was maintained at 35° C. Mobile phase A was 0.1% formic acid in water, and mobile phase B was acetonitrile. Gradient elution was performed as follows: 0 min, 20% B; 0-4.5 min, 20%-35% B; 4.5-6 min, 35%-50% B; 6-12 min, 50%-65% B; 12-18 min, 65%-85% B; 18-22.5 min, 85%-95% B; 22.5-24 min, 95%-20% B; and 24-30 min, 20% B. The flow rate was 0.2 mL/min, and the injection volume was 1 μL. Qualitative and quantitative detection were performed using the external standard method. The detection results confirmed the production of 0.88 mM vestitone.

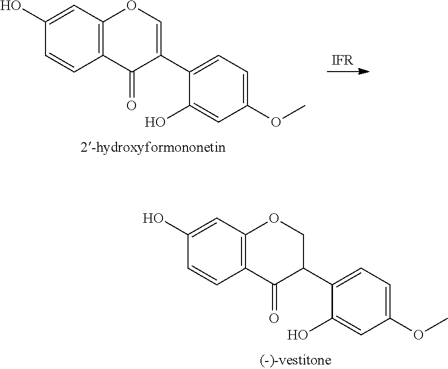 [SMILES: COc1ccc(-c2coc3cc(O)ccc3c2=O)c(O)c1 ; "IFR" ; COc1ccc(C2COc3cc(O)ccc3C2=O)c(O)c1]

### Example 4: Expression and Functional Verification of Vestitone Reductase

Vestitone reductase derived from Glycyrrhiza glabra (GgVR), with the amino acid sequence of SEQ ID NO: 8 and the nucleotide sequence of SEQ ID NO: 20 accordingly, was selected and subjected to gene synthesis according to the sequence provided. The synthesized gene was amplified by PCR and ligated into the Saccharomyces cerevisiae expression vector pAK203 using Gibson assembly to obtain a recombinant plasmid. The recombinant plasmid was introduced into Escherichia coli competent cells DH5a by electroporation. Escherichia coli clones transformed with the expression plasmid successfully were picked and cultured in 1 mL of LB medium containing the corresponding antibiotics at 37° C. with shaking at 220 rpm for 12 hours. The bacterial cells were collected by centrifugation to extract the plasmid. The plasmid was transformed into Streptomyces coelicolor cells by protoplast transformation. The positive clones obtained after transformation were inoculated into medium and cultured at 30° C. with shaking at 250 rpm for 24 hours. Then, thiostrepton was added for induction, and 1 mM vestitone was added as the reaction substrate at the same time. The reaction was carried out at 30° C. with shaking at 220 rpm for 24-48 hours, and samples were collected for product detection.

The reacted culture solution was centrifuged, and the supernatant was collected and extracted with an equal volume of ethyl acetate. The obtained organic phase was evaporated to dryness and redissolved with methanol. The production of medicarpin was detected by UPLC or LC-MS.

Specifically, quantitative detection of the fermentation products from the engineered strains was performed using a UPLC instrument (Agilent 1260). Separation was carried out on a Poroshell 120 EC-C18 column of particle size 2.7 μm and 3.0×100 mm. The detection wavelength was set at 254 nm, and the column oven temperature was maintained at 35° C. Mobile phase A was 0.1% formic acid in water, and mobile phase B was acetonitrile. Gradient elution was performed as follows: 0 min, 20% B; 0-4.5 min, 20%-35% B; 4.5-6 min, 35%-50% B; 6-12 min, 50%-65% B; 12-18 min, 65%-85% B; 18-22.5 min, 85%-95% B; 22.5-24 min, 95%-20% B; and 24-30 min, 20% B. The flow rate was 0.2 mL/min, and the injection volume was 1 μL. Qualitative and quantitative detection were performed using the external standard method. The detection results confirmed the production of 0.9 mM medicarpin.

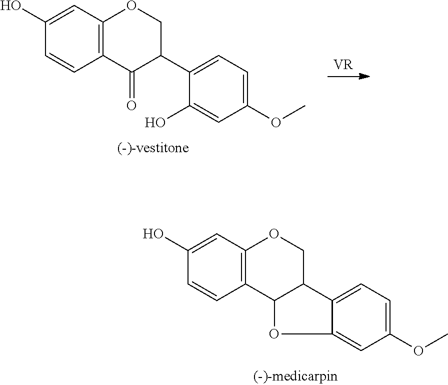

### Example 5: Expression and Functional Verification of Prenyltransferase, Pterocarpan Reductase, Oxidative Cyclase, and Demethylase

Prenyltransferase derived from Glycyrrhiza glabra (GgPT) with the amino acid sequence of SEQ ID NO: 10, pterocarpan reductase derived from Glycyrrhiza glabra (GgPTR) with the amino acid sequence of SEQ ID NO: 11, oxidative cyclase derived from Glycyrrhiza glabra (GgOC) with the amino acid sequence of SEQ ID NO: 13, and demethylase derived from Glycyrrhiza glabra (GgDMT) with the amino acid sequence of SEQ ID NO: 15 were selected and subjected to gene synthesis according to the sequences provided. Their corresponding nucleotide sequences are SEQ ID NO: 21, SEQ ID NO: 22, SEQ ID NO: 23, and SEQ ID NO: 24 in sequence. The synthesized genes were amplified by PCR and ligated into the Agrobacterium expression vector pBI121 using Gibson assembly to obtain a recombinant plasmid. The recombinant plasmid was introduced into Agrobacterium competent cells EHA105 by electroporation. Agrobacterium clones successfully transformed with the expression plasmid were picked and cultured in 1 mL of LB medium containing the corresponding antibiotics at 28° C. with shaking at 250 rpm for 24 hours. Then, 50 μl of cultured Agrobacterium solution was inoculated into 5 ml of LB medium containing the corresponding antibiotics as well as with 100 μl of 0.5 M MES and 2 μl of 100 mM AS added, and then further cultured at 28° C. with shaking at 250 rpm until OD600=1.0. A centrifugation at 4000 rpm for 10 minutes was performed, and the precipitate of bacterial cells was collected and resuspended in 10 mM MgCl₂ to OD600=1.0. Then, 2 μl of 100 mM AS was added per ml of bacterial resuspended solution, followed by standing for more than 3 hours.

Nicotiana benthamiana in the vigorous growth period was selected. The bacterial resuspension was drawn up into a syringe without a needle; with fingers pressing against the front of the leaf, the bacterial solution was infiltrated from the back of the leaf, and 1 mM medicarpin solution was injected as the reaction substrate at the same time. Samples were collected for product detection after 24-48 hours of placement.

The collected leaves were cut into pieces and subjected to bead-beating disruption at −20° C. After disruption, the precipitate and the separated supernatant were extracted with an equal volume of ethyl acetate. The obtained organic phase was evaporated to dryness and redissolved with methanol. The production of glabridin was detected by UPLC or LC-MS.

Specifically, quantitative detection of the fermentation products from the engineered strains was performed using a UPLC instrument (Agilent 1260). Separation was carried out on a Poroshell 120 EC-C18 column of particle size 2.7 μm and 3.0×100 mm. The detection wavelength was set at 254 nm, and the column oven temperature was maintained at 35° C. Mobile phase A was 0.1% formic acid in water, and mobile phase B was acetonitrile. Gradient elution was performed as follows: 0 min, 20% B; 0-4.5 min, 20%-35% B; 4.5-6 min, 35%-50% B; 6-12 min, 50%-65% B; 12-18 min, 65%-85% B; 18-22.5 min, 85%-95% B; 22.5-24 min, 95%-20% B; and 24-30 min, 20% B. The flow rate was 0.2 mL/min, and the injection volume was 1 μL. Qualitative and quantitative detection were performed using the external standard method. The detection results confirmed the production of glabridin.

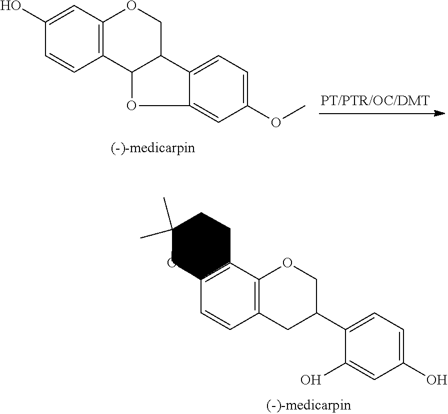

### Example 6: Construction and Verification of the Pathway for Synthesizing Glabridin Using Daidzein as Substrate

4′-O-methyltransferase derived from Pueraria lobata (PlOMT9) with the amino acid sequence of SEQ ID NO: 1, isoflavone 2′-hydroxylase derived from Glycyrrhiza echinata (GeCYP81E1) with the amino acid sequence of SEQ ID NO: 3, isoflavone reductase derived from Medicago sativa (MsIFR) with the amino acid sequence of SEQ ID NO: 5, vestitone reductase derived from Medicago sativa (MsVR) with the amino acid sequence of SEQ ID NO: 7, pterocarpan prenyltransferase derived from Cullen corylifolium (tPcM4DT) with the amino acid sequence of SEQ ID NO: 16, pterocarpan reductase derived from Glycyrrhiza glabra (GgPTR) with the amino acid sequence of SEQ ID NO: 11, oxidative cyclase derived from Glycyrrhiza glabra (GgOC) with the amino acid sequence of SEQ ID NO: 13, and demethylase derived from Nectria haematococca (NhDMT) with the amino acid sequence of SEQ ID NO: 14 were selected and subjected to gene synthesis according to the sequences provided. The corresponding nucleotide sequences of the enzymes are SEQ ID NO: 25, SEQ ID NO: 26, SEQ ID NO: 27, SEQ ID NO: 28, SEQ ID NO: 29, SEQ ID NO: 30, SEQ ID NO: 31, and SEQ ID NO: 32 in sequence, respectively. The synthesized genes were amplified by PCR, ligated into an expression cassette using Overlap Extension-PCR (OE-PCR) and Gibson assembly, and constructed into the commonly used vector plasmid pYL15 (GenBank accession: KU378202) for Yarrowia lipolytica. The expression cassette sequence was: PTDH-PlOMT9-TVMA-PFBA-GmCYP81E1-TCYC-PTEF-MsIFR-TCDC-PTEF-MsVR-TATP-PENO-tPcM4DT-TADH-PTDH-GgPTR-TRPL-PHSP12-GgOC-TSOD1-PHSP26-NhDMT-TFBP-URA3. The recombinant plasmid was introduced into Escherichia coli competent cells DH5a by transformation. Escherichia coli clones successfully transformed with the expression plasmid were picked and cultured in 1 mL of LB medium containing the corresponding antibiotics at 37° C. with shaking at 220 rpm for 12 hours. The bacterial cells were collected by centrifugation to extract the plasmid. The plasmid was transformed into Yarrowia lipolytica ATCC201249. The positive clones obtained after transformation were inoculated into SD-Ura medium and cultured at 30° C. with shaking at 250 rpm for 16 hours. Then, 5 mM daidzein was added as the reaction substrate, and the reaction was carried out at 30° C. with shaking at 220 rpm for 96 hours, then samples were collected for product detection.

The reacted culture was subjected to bead-beating disruption and extracted with an equal volume of ethyl acetate. The obtained organic phase was evaporated to dryness and redissolved with methanol. The production of glabridin was detected by UPLC or LC-MS.

Specifically, quantitative detection of the fermentation products from the engineered strains was performed using a UPLC instrument (Agilent 1260). Separation was carried out on a Poroshell 120 EC-C18 column of particle size 2.7 μm and 3.0×100 mm. The detection wavelength was set at 254 nm, and the column oven temperature was maintained at 35° C. Mobile phase A was 0.1% formic acid in water, and mobile phase B was acetonitrile. Gradient elution was performed as follows: 0 min, 20% B; 0-4.5 min, 20%-35% B; 4.5-6 min, 35%-50% B; 6-12 min, 50%-65% B; 12-18 min, 65%-85% B; 18-22.5 min, 85%-95% B; 22.5-24 min, 95%-20% B; and 24-30 min, 20% B. The flow rate was 0.2 mL/min, and the injection volume was 1 μL. Qualitative and quantitative detection were performed using the external standard method. The detection results confirmed the production of 1.8 mM glabridin.

### Example 7: Construction and Verification of the Pathway for Synthesizing Glabridin Using Daidzein as Substrate

4′-O-methyltransferase derived from Glycyrrhiza glabra (GgOMT) with the amino acid sequence of SEQ ID NO: 2, isoflavone 2′-hydroxylase derived from Glycyrrhiza glabra (GgI2′H) with the amino acid sequence of SEQ ID NO: 4, isoflavone reductase derived from Glycyrrhiza glabra (GgIFR) with the amino acid sequence of SEQ ID NO: 6, vestitone reductase derived from Glycyrrhiza glabra (GgVR) with the amino acid sequence of SEQ ID NO: 8, prenyltransferase derived from Glycyrrhiza glabra (GgPT) with the amino acid sequence of SEQ ID NO: 10, pterocarpan reductase derived from Glycyrrhiza glabra (GgPTR) with the amino acid sequence of SEQ ID NO: 12, oxidative cyclase derived from Glycyrrhiza glabra (GgOC) with the amino acid sequence of SEQ ID NO: 13, and demethylase derived from Glycyrrhiza glabra (GgDMT) with the amino acid sequence of SEQ ID NO: 15 were selected and subjected to gene synthesis according to the sequences provided. The corresponding nucleotide sequences of the enzymes are SEQ ID NO: 33, SEQ ID NO: 34, SEQ ID NO: 19, SEQ ID NO: 35, SEQ ID NO: 36, SEQ ID NO: 37, SEQ ID NO: 38, and SEQ ID NO: 39 in sequence, respectively. The synthesized genes were amplified by PCR, ligated into the expression cassette of HOL-PTDH3-GgOMT-TVMA1-PFBA1-GgI2′H-TCYC1-PTEF1-GgIFR-TCDC19-PTEF2-GgVR-TATP1-PENO2-GgPT-TADH1-PTDH1-GgPTR-TRPL1-PHSP12-GgOC-TSOD1-PHSP26-GgDMT-TFBP1-URA3-HOR using OE-PCR and Gibson assembly, and further ligated into the vector pUC19. The recombinant plasmid was introduced into Escherichia coli competent cells DH5a by transformation. Escherichia coli clones successfully transformed with the expression plasmid were picked and cultured in 1 mL of LB medium containing the corresponding antibiotics at 37° C. with shaking at 220 rpm for 12 hours. The bacterial cells were collected by centrifugation to extract the plasmid. DNA fragments \textcircled{1} HOL-PTDH3-GgOMT-TVMA1-PFBA1-GgI2′H-TCYC1-PTEF1-GgIFR-TCDC19, \textcircled{2} TCDC19-PTEF2-GgVR-TATP1-PENO2-GgPT-TADH1-PTDH1-GgPTR-TRPL1-PHSP12, \textcircled{3} PHSP12-GgOC-TSOD1-PHSP26-GgDMT-TFBP1-URA3-HOR were amplified by PCR, respectively, and then simultaneously transformed into Saccharomyces cerevisiae BY4741. The positive clones obtained after transformation were inoculated into SD-Ura medium and cultured at 30° C. with shaking at 250 rpm for 16 hours. Then, 1 mM daidzein was added as the reaction substrate, and the reaction was carried out at 30° C. with shaking at 220 rpm for 96 hours, then samples were collected for product detection.

The reacted culture was subjected to bead-beating disruption and extracted with an equal volume of ethyl acetate. The obtained organic phase was evaporated to dryness and redissolved with methanol. The production of glabridin was detected by UPLC or LC-MS.

Specifically, quantitative detection of the fermentation products from the engineered strains was performed using a UPLC instrument (Agilent 1260). Separation was carried out on a Poroshell 120 EC-C18 column of particle size 2.7 μm and 3.0×100 mm. The detection wavelength was set at 254 nm, and the column oven temperature was maintained at 35° C. Mobile phase A was 0.1% formic acid in water, and mobile phase B was acetonitrile. Gradient elution was performed as follows: 0 min, 20% B; 0-4.5 min, 20%-35% B; 4.5-6 min, 35%-50% B; 6-12 min, 50%-65% B; 12-18 min, 65%-85% B; 18-22.5 min, 85%-95% B; 22.5-24 min, 95%-20% B; and 24-30 min, 20% B. The flow rate was 0.2 mL/min, and the injection volume was 1 μL. Qualitative and quantitative detection were performed using the external standard method. The detection results confirmed the production of 0.6 mM glabridin.

The enzymes, biological hosts, substrates, and products described in the examples above are shown in Table 3 below. It can be seen from Table 2 that the series of enzymes for synthesizing glabridin derived from Glycyrrhiza glabra, which are first disclosed in the present disclosure, exhibit respective activities in various biological hosts. In addition, for the first time, the heterologous biosynthesis of glabridin is realized, with a yield of glabridin reaching over 6000.

TABLE 2

| | | Chassis | | |
|---|---|---|---|---|
| | Enzyme And Origin | Biological | | |
| Example | Thereof | Host | Substrate | Product |
| Example 1 | 4′-O-methyltransferase | wild-type | 1 mM | 4.5 mg |
| | derived from | Nicotiana | daidzein | formononetin/ |
| | Glycyrrhiza glabra | benthamiana | | g |
| | (GgOMT), SEQ ID NO: | | | Nicotiana |
| | 2 | | | species |
| Example 2 | Isoflavone 2′- | Escherichia | 1 mM | 0.96 mM |
| | hydroxylase derived | coli BL21 | formononetin | 2′- |
| | from Glycyrrhiza glabra | | | hydroxyformononetin |
| | (GgI2′H), SEQ ID NO: 4 | | | |
| Example 3 | Isoflavone reductase | Saccharomyces | 1 mM 2′- | 0.88 mM |
| | derived from | cerevisiae | hydroxyformononetin | vestitone |
| | Glycyrrhiza glabra | INVSC1 | | |
| | (GgIFR), SEQ ID NO: 6 | | | |
| Example 4 | Vestitone reductase | Streptomyces | 1 mM | 0.9 mM |
| | derived from | coelicolor (S. | vestitone | medicarpin |
| | Glycyrrhiza glabra | coelicolor) | | |
| | (GgVR), SEQ ID NO: 8 | A3(2) | | |
| Example 5 | Prenyltransferase | wild-type | 1 mM | 7.3 mg |
| | derived from | Nicotiana | medicarpin | glabridin/ |
| | Glycyrrhiza glabra | benthamiana | | g |
| | (GgPT), SEQ ID NO: 10; | | | Nicotiana |
| | pterocarpan reductase | | | species |
| | derived from | | | |
| | Glycyrrhiza glabra | | | |
| | (GgPTR), SEQ ID NO: | | | |
| | 11; | | | |
| | oxidative cyclase | | | |
| | derived from | | | |
| | Glycyrrhiza glabra | | | |
| | (GgOC), SEQ ID NO: | | | |
| | 13; | | | |
| | demethylase derived | | | |
| | from Glycyrrhiza glabra | | | |
| | (GgDMT), SEQ ID NO: | | | |
| | 15 | | | |
| Example 6 | 4′-O-methyltransferase | Yarrowia | 5 mM | 1.8 mM |
| | derived from Pueraria | lipolytica | daidzein | glabridin |
| | lobata (PlOMT9) , SEQ | ATCC 201249 | | |
| | ID NO: 1; | | | |
| | isoflavone 2′- | | | |
| | hydroxylase derived | | | |
| | from Glycyrrhiza | | | |
| | echinata (GeCYP81E1), | | | |
| | SEQ ID NO: 3; | | | |
| | isoflavone reductase | | | |
| | derived from Medicago | | | |
| | sativa (MsIFR), SEQ ID | | | |
| | NO: 5; | | | |
| | vestitone reductase | | | |
| | derived from Medicago | | | |
| | sativa (MsVR), SEQ ID | | | |
| | NO: 7; | | | |
| | pterocarpan | | | |
| | prenyltransferase | | | |
| | derived from Cullen | | | |
| | corylifolium | | | |
| | (tPcM4DT), SEQ ID | | | |
| | NO: 16; | | | |
| | pterocarpan reductase | | | |
| | derived from | | | |
| | Glycyrrhiza glabra | | | |
| | (GgPTR), SEQ ID NO: | | | |
| | 11; | | | |
| | oxidative cyclase | | | |
| | derived from | | | |
| | Glycyrrhiza glabra | | | |
| | (GgOC), SEQ ID NO: | | | |
| | 13; | | | |
| | demethylase derived | | | |
| | from Nectria | | | |
| | haematococca | | | |
| | (NhDMT), SEQ ID NO: | | | |
| | 14 | | | |
| Example 7 | 4′-O-methyltransferase | Saccharomyces | 1 mM | 0.6 mM |
| | derived from | cerevisiae | daidzein | glabridin |
| | Glycyrrhiza glabra | BY4741 | | |
| | (GgOMT), SEQ ID NO: | | | |
| | 2; | | | |
| | isoflavone 2′- | | | |
| | hydroxylase derived | | | |
| | from Glycyrrhiza glabra | | | |
| | (GgI2′H), SEQ ID NO: | | | |
| | 4; | | | |
| | isoflavone reductase | | | |
| | derived from | | | |
| | Glycyrrhiza glabra | | | |
| | (GgIFR), SEQ ID NO: | | | |
| | 6; | | | |
| | vestitone reductase | | | |
| | derived from | | | |
| | Glycyrrhiza glabra | | | |
| | (GgVR), SEQ ID NO: 8; | | | |
| | prenyltransferase | | | |
| | derived from | | | |
| | Glycyrrhiza glabra | | | |
| | (GgPT), SEQ ID NO: 10; | | | |
| | pterocarpan reductase | | | |
| | derived from | | | |
| | Glycyrrhiza glabra | | | |
| | (GgPTR), SEQ ID NO: | | | |
| | 12; | | | |
| | oxidative cyclase | | | |
| | derived from | | | |
| | Glycyrrhiza glabra | | | |
| | (GgOC), SEQ ID NO: | | | |
| | 13; | | | |
| | demethylase derived | | | |
| | from Glycyrrhiza glabra | | | |
| | (GgDMT), SEQ ID NO: | | | |
| | 15 | | | |

Sequence Information

>SEQ ID NO: 1 (PIOMT9)
MGSNNGRKASEIFQGQALLYRHMFAFVDSMCLKSIVELGIPNIIHKHGQP
ITLSELMSILQVPPAKVGHVQSLMRYLAHHGFFERLRIHEKDAYALTAAS
ELLVKGTEPYLAPMVECMLDPTLSASFHQMKKWVYEEDLSVFDISLGS
NLWDFLTKNPAYNEIFNEAMASDSQMSNLALRDCKLVFEGLESIVDVGG
GTGTTAKIICEAFPNLKCIVFDRPQVVENLLENNNLTYVGGDMFKSIPKA
NAVLLKWILHDWTDKDCRKILENCKEAISNNSKRGKIIIIDIVINEKQDEH
KVTELKLLFDVAMACVLNGKERNEEEWKKLFMEVGLQDYKISPLTGYLSLIEIYP*
>SEQ ID NO: 2 (GgOMT)
MDFSTNGSEESELYHAQIHLYKHVYNFVSSMALKSAMELGIADVIHNH
GKPITLPQLASALKLHPSKVGVLYRFLRLLTHNGFFAKTTVPSQNGKEGE
EEETAYALTPPSKLLVKGKPTCLASIVRGALHPSSLDMWRSSEKWFNED
KELTLFESATGESFWDFLNKDSESGTLSMFQEAMAADSQMFKLALKEC
RHVFEGLESLVDVGGGTGGVTKLIHEEFPHLKCTVFDQPQVVGNLSGN
ENLKFVGGDMFKSIPPADAVLLKWVLHDWNDELSLKILKNSKEAISGKG
KEGKVIIIDISIDEASGDRELTELQLDYDLVMLTMFNGKEREKKEWEKLI
SDAGFSSYKITPICGFKSLIEVFP*
>SEQ ID NO: 3 (GeCYP81E1)
MEILSLLSYSVFYLALFFIFNIVIRARKFKNLPPGPPSLPIIGNLHHLKRPL
HRTFKGLSEKYGHVFSLWFGSRLVVVVSSASEFQQCFTKNDVVLANRP
RFLSGKYIFYNYTTLGSTSYGEHWRNLRRITALDVLSNHRINSFSGIRRD
ETQRLITRLADDSSTNFAEMELSSRLYDMTFNNIMRMISGKRYYGEDCD
TSDLQEASQFRDMVSELLQLSGANNKTDFMPLLRFLDFENLEKRLKDIS
GKTDAFLRGLIEEHRTKKERANTMIDHLLNLQDSQPEYYTDQIIKGLAL
AMLLAGTDSSAVTLEWSMSNLLNHPEVLKKVKDELDTHVGQDRLVDE
SDLPKLTYLKNVINETLRLYTPAPLLLPHSTSDECNIGGYKVPQDTIVLIN
AWAIHRDPELWTEATTFKPERFEKKGELEKLIAFGMGRRACPGEGLAIR
AISMTLALLIQCFDWKLINGDKIDLAERDGFTLTKLVPLKAMCKSRPVINKVFKQ*
>SEQ ID NO: 4 (GgI2′H)
MEILSLLSYSVFYVALFFICNLLFQARKFKNLPPGPPSLPIIGNLHHLKRPL
HRTFKGLSEKYGHVISLWFGSRLVVVVSSLSVFQECFTKNDIVLADRPRF
LSGKYIFYNYTTVGSSSYGEHWRNLRRITSLDVLSTHRINSFSGVRRDET
QRLIQKLAEESSTDFAEIELTSKFYDMTFNNIMRMISGKRYYGDDCDMM
DMEEAKQFRAMVSELLQLSGANNKTDFMPVLRLVDFENLEKRLKKISS
KTDTFLRGLIQEHRNKKQHTNTMVDHLLSLQESQPEYYTDQIIKGLALG
MLLAGTDSSAVTLEWALSCLLNHPEALKKARDELETHVGQDRLLEESD
LTKLPYLKNIIYETLRLYTPAPLLLPHSSSDECIIGGFKVPRETIILINAWSIH
RDPQIWSEATSFKPERFEKEGELDKLIAFGLGRRACPGEALALRGISLTL
GLLIQCFEWKLVGDKEIDMREESGFTLSRLIPLKAMCKARPVANKLVNTQAVSLGD*
>SEQ ID NO: 5 (MsIFR)
MATENKILILGPTGAIGRHIVWASIKAGNPTYALVRKTPGNVNKPKLITA
ANPETKEELIDNYQSLGVILLEGDINDHETLVKAIKQVDIVICAAGRLLIE
DQVKIIKAIKEAGNVKKFFPSEFGLDVDRHDAVEPVRQVFEEKASIRRVI
EAEGVPYTYLCCHAFTGYFLRNLAQLDATDPPRDKVVILGDGNVKGAY
VTEADVGTFTIRAANDPNTLNKAVHIRLPKNYLTQNEVIALWEKKIGKT
LEKTYVSEEQVLKDIQESSFPHNYLLALYHSQQIKGDAVYEIDPAKDIEASEAYPDVTYT
TADEYLNQFV*
>SEQ ID NO: 6 (GgIFR)
MNMIEVGGYPGNPINTKQSNAQQHKPTASLTTYQNKKKKNTHPSFFNSP
YFYFNFNIPMAAENKILILGPTGAIGRHIVWASVKAGNPTFALVRKTNGP
VNKPKLITAANPESKEQLLESYQNAGVTLLEGDINDHESLVKAIKQVDV
VICATGRLLIDDQVKIIAAIKEAGNVKRFFPSEFGLDVDRHDSVEPVREV
FEDKARIRRVIEAEGVPYTYLCCHAFTGYFLRNLAQLDATVPPRDKVVIL
GDGNVKGAYVTEADVGTYTIRAANDPRTLNKAVHIRLPANYLTANEVV
SLWEKKIGKTLEKTYVPEEKVLKDIQESSFPHNYLLALYHSQQIKGDAV
YEIDPAKDVEAYDLYSDVKYTTADEYLNQFV*
>SEQ ID NO: 7 (MsVR)
MAEGKGRVCVTGGTGFLGSWIIKSLLENGYSVNTTIRADPERKRDVSFL
TNLPGASEKLHFFNADLSNPDSFAAAIEGCVGIFHTASPIDFAVSEPEEIVT
KRTVDGALGILKACVNSKTVKRFIYTSSGSAVSFNGKDKDVLDESDWS
DVDLLRSVKPFGWNYAVSKTLAEKAVLEFGEQNGIDVVTLILPFIVGRFV
CPKLPDSIEKALVLVLGKKEQIGVTRFHMVHVDDVARAHIYLLENSVPG
GRYNCSPFIVPIEEMSQLLSAKYPEYQILTVDELKEIKGARLPDLNTKKLVDAGFDFKYTIE
DMFDDAIQCCKEKGYL*
>SEQ ID NO: 8 (GgVR)
MAEGKGRVCVTGGTGFLGSWLIKRLLEDGYGVNTTIRSDPERKRDISFL
TNLPGASERLKIFNADLSDPESFGPAIEGCVGVFHTASPIDFAVSEPEEIVT
KRTVDGTLGILKACKNSKTVKRVVYTSSGAAVSWGGTEKDVLDESDW
SDVDMLRSVKPYSWSYAVSKTMTEKAVLEFGEQHGLDVVTLILPFIVGR
FICPKIPDSVEKALVLVLGKREQIGVTRFHMVHVEDVARAYIFLLEHPNP
KGRYNCSPFIVPIEEVSELLSAKYPEYQIPTVEELKEIKGAKLPDLKSKKLVDAGFEFKYTI
EDMFDDAIQCCKEKGYL*
>SEQ ID NO: 9 (PcM4DT)
MDWGLAISSSSKAYSVTTGANLWRSKHVTNNIHYPSSCITKASQHKKKT
QIEYNVLRFQRPSLGHGYNFSGGGGSTYQECNRKYAVKAVYDQPKDFE
LEASNPKNILDSAKKFLAAFYYFSYPYTMIGITLCAFCSSALAVEKLSDIS
LPFFIGVLQAVIPQLFIEIYLSGVNQLYDLEIDKINKPHLPMASGQFSFKTG
VIMSAAFLALSFGFTWLTGSWPLIWNLVVIASSWTAYSIDVPFMRWKRY
PLVAAMCMIATWGLALPISFFHHMQTFVLQRPIGFPRSLGFLVAFMTFYS
MGIALSKDIPDVEGDKEHGINSFSVLLGQKRIFWICVSLFEMAFGVGLLG
GVTSPHFWTKIITGLGNVILGSILWYQAKSFDLTDKASTGSFYMYIWKLLYAALLLMAFVR*
>SEQ ID NO: 10 (GgPT)
MSSLFGSNVFESISTYRSYAPKASQHKRKIQKEYNFLRSRQPSLKHIYKGI
EGGSTQQECNRKYVVKTVPKPSFESDPRSIDQKDILECIKNFLDALYMFT
TPYSFFSSALYIISVSFIVVEKLSDISPLFFTGVLQAVVPNLFVHIYMTGLN
QLCDVEIDKINKPYLPLVSGKISFATGVIVVASCLFLSLWLGWIVGSWPST
CVVISIAMIWTAYSINVPLLRWKKYPVLAAMCIFLSLAVVNPIGYFLHMQ
TFVFKRPASFSRPLIFVIAFKSFFSLAVALFKDVPDIEGDQTFGVQSFVARF
GKKRVFWICISLIEMAYGFALLMGATSAFLWSKIVTVLGHAVLASFVLYR
AKSIDLRSKTSITSFYMLIWKLMYMEYFLMPLVR*
>SEQ ID NO: 11 (GgPTR)
MAETTTSKSKILFIGGTGYIGKFIVEASVNAGHPTFVLIRDSTLSNPAKSPI
IDKFKSLAVNLVFGDLYDHQSLVKAIKQVDVVISTVGHLQLGDQDKIISA
IKESGNVKRFFPSEFGNDVDRTHAVEPAKSAFATKAKIRRTIEAEGIPYTY
VSSNFFAGYFLPTLSQPGATAAPRDKVIILGDGNPKAVANKEEDIATYTIK
AVDDPRTLNKILYIRPPANTLSFNELVSLWEKKIGKTLGRVYVPEEQLLK
QIQESSPPINVILSIGHSVYVKGDHTNFEIEPAFGVEASALYPDVKYTTVDEYLNQFV*
>SEQ ID NO: 12 (GgPTR)
MDSTAASKILVIGGTGYMGRFLVEASAKAGHPTFALVRHSTVTNPDKSSI
IHSFNTLGVNLLLGDINDHQSLVKAIKQADVVISTVNHQYISDQYKIISAI
KEAGNIKRFFPSEFGNDVVRTHGVDGAKALFDIKAKFRRTIEAEGIPHTY
VVANFLTQHFLPTRSRLLAIAAPLDKVVILGDGNTKATFNTEEGVATFTIR
AVDDPRTLNKILYIRPPANTLSYNDLVSLWEKKTGNTLERVYVPEEQVLR
LIQESSYPLNMALSICHAAYVKGDHTNYEIEPSFGVEALELYPDVKFTTV
DDYLEQNRDCTPFYLNQLIPITNGTKF*
>SEQ ID NO: 13 (GgOC)
MAFTYSTKKLALLSIITIFISSFPEPSTSVDLESGFLQCFSSGLGNSNSTAEL
VLTKNSSSYASLLQSSIRNLRFMGTSVPKPTLIVTPRDLFHIQTSIKCSVK
QGLQIRVRSGGHDYEGLSYVSNDDVPFLIIDLTNLRSITIDIKDETAWVQS
GATLGELYYAIAKKSNVHGFPAGSCPTVGVGGHFSGGGFGTIFRKYGLA
ADNVIDAQMVDVNGKILKNRTLMGEDLFWAIRGGGGSSFGVVTAWKV
KLVHVPPKVTVFNIPKTLDQNASTLFHKWQIVADKLPGELFLHSVMGVS
NANSISSSSGGGRTVLVSFTGLYLGTVENLLPLMQNNFAELGLQHNNCT
EMSWIQSVLFFAGYDPINDSLEVLLQRNQTFGSFKAKSDYVMEPIPTSGL
EGLWNMLLEENSSPTLILTPYGGRMSEISESETPFPHRNGSIYGIQYLVY
WDSSEETPKHIAWMRRVYSYMVLYVSKFPRAAYLNYRDLDIGVNRGNT
SYDYEEAKSWGLKYFKCNFERLAKVKAEVDPSNFFRHEQSIPLLF*
>SEQ ID NO: 14 (NhDMT)
MLVDTGLGLISELQAKLGWAVLLQIVPITIVAYNLLWFIYASFFSSLRKIPG
PFLARISRVWEMKKTATGNIHEIMMDLHRRHGAIVRIGPRRYDFDTMEA
LKIIYRIGNALPKADYYKPFGLPSFPNLFDEQNPARHSAIKKQVASLYTM
TALLSYEEGVDGQTAILKEQLQRFCDQKQVIDLPRFLQYYAFDVIGVITV
GKSMGMMESNSDTNGACSALDGMWHYASMMAYIPNMHAWWLRLSS
LLPIEVPIKGLTEYVERRIIQYRLKAAEFGDDAALKGENNFLAKLLLMEK
KGTVTPVETQQAVGLNIGAGSDTTANALSTILYYLYTNPRTLHTLREELE
RYVKDGPISFQQSQSMPYLQAVIKEALRLHPGVGTQLTRVVPKGGLVIEG
QFFPEGTEVGVNGWALYHNKAIFGNDASIFRPERWLEANENINIGGSFAF
GAGSRSCIGKNISILEMSKAIPQIVRNFDIEINHGDMTWKNECWWFVKPE
YKAMIKPRRCCLSRDESLV*
>SEQ ID NO: 15 (GgDMT)
MEHFYMSLLLLFVTLVSLSLFFLIFYHNKHNMNNNNNLPPGKMGYPVIG
ESLEFLSMGWKGHPEKFIFDRMVRYSSELIKTSILGVPTVIFCGPACNKFL
FSNENKLVTAWWPDSVNKIFPTTSNSKEESKKMRKLLPQFLKPEALQRY
VGIMDTLAQRHFASLWEEKTHVTVYPLAKRYTFMLACRLFMSVEDENH
VAKFREPFHLLASGIISVPIDLPWTPFNRGIKASNFIRKELLKIIRQRKVDL
AQGVASPTQDILSHMLLTCDDENGEFMTELNIADKILGLLIGGHDTASAA
CTFIVKYLAELPHIYDRVYQEQMEIANSKSPGELLNWDDINKMRYSWN
VASEVMRVAPPLQGGFREAINDFVFNGFSIPKGWKLYWSANSTHKNPEY
FPAPEKFDPTRFEGNGPAAYTFVPFGGGPRMCPGKEYARLEILVFMHNLV
KRFKWEMLIPEEKIVVDPLPMPANDLPIRLYPHNT*
>SEQ ID NO: 16 (tPcM4DT)
ASQHKKKTQIEYNVLRFQRPSLGHGYNFSGGGGSTYQECNRKYAVKAV
YDQPKDFELEASNPKNILDSAKKFLAAFYYFSYPYTMIGITLCAFCSSAL
AVEKLSDISLPFFIGVLQAVIPQLFIEIYLSGVNQLYDLEIDKINKPHLPMA
SGQFSFKTGVIMSAAFLALSFGFTWLTGSWPLIWNLVVIASSWTAYSIDV
PFMRWKRYPLVAAMCMIATWGLALPISFFHHMQTFVLQRPIGFPRSLGF
LVAFMTFYSMGIALSKDIPDVEGDKEHGINSFSVLLGQKRIFWICVSLFE
MAFGVGLLGGVTSPHFWTKIITGLGNVILGSILWYQAKSFDLTDKASTG
SFYMYIWKLLYAALLLMAFVR*
>SEQ ID NO: 17 (GgOMT)
ATGGACTTTTCGACAAATGGGTCTGAGGAAAGTGAACTTTATCATGCT
CAAATTCACTTGTACAAGCATGTTTACAATTTTGTGTCTTCAATGGCTT
TAAAGTCAGCCATGGAGTTGGGCATAGCTGATGTCATACACAATCATG
GTAAGCCAATCACCCTCCCCCAGCTGGCATCTGCTCTGAAATTGCACC
CTTCTAAGGTTGGAGTACTTTACCGATTCCTCCGGTTGCTAACTCACA
ACGGCTTCTTTGCTAAAACCACTGTTCCTTCTCAGAATGGAAAAGAA
GGCGAGGAGGAGGAAACGGCTTATGCATTGACTCCACCATCAAAATT
ATTGGTAAAAGGAAAGCCTACTTGTTTGGCGAGCATTGTTAGAGGTG
CACTGCATCCTAGTAGCCTTGACATGTGGAGAAGTTCCGAGAAGTGG
TTCAATGAAGATAAAGAGCTTACGTTATTCGAGTCAGCCACAGGAGA
AAGTTTTTGGGATTTCTTGAACAAGGACAGCGAATCAGGAACTCTCT
CAATGTTTCAAGAAGCAATGGCAGCAGACTCTCAAATGTTCAAACTT
GCCTTGAAGGAATGCAGGCATGTGTTTGAGGGTTTGGAATCTCTTGTT
GATGTGGGTGGAGGAACTGGAGGGGTTACCAAGTTAATCCATGAAGA
ATTTCCCCATCTGAAATGCACAGTATTTGATCAACCGCAAGTAGTGGG
GAACCTAAGTGGAAATGAGAATCTGAAGTTTGTTGGTGGTGATATGTT
TAAGAGTATACCACCTGCTGATGCTGTCCTACTGAAATGGGTTCTTCA
TGATTGGAATGATGAGCTTAGCCTCAAAATCCTTAAAAACTCCAAGG
AGGCTATTTCTGGTAAAGGCAAAGAAGGGAAAGTGATAATCATTGAC
ATATCGATTGATGAAGCGTCAGGGGATAGGGAACTTACAGAATTGCA
GTTGGATTATGATCTAGTGATGCTGACAATGTTCAATGGTAAGGAGCG
TGAGAAAAAGGAATGGGAAAAGCTCATTAGTGACGCAGGATTTTCCT
CATATAAAATTACACCAATTTGTGGTTTCAAATCTTTAATTGAGGTCTTTCCTTGA
>SEQ ID NO: 18 (GgI2′H)
ATGGAAATATTGTCACTATTAAGTTATAGCGTGTTCTACGTGGCCCTGT
TTTTCATCTGCAACCTGTTGTTCCAAGCTCGCAAATTCAAAAACCTGC
CACCGGGTCCGCCAAGCCTGCCTATCATCGGTAATCTTCACCATCTGA
AGCGTCCGCTGCACCGCACCTTCAAGGGTTTGTCCGAAAAGTACGGC
CACGTGATTAGCTTATGGTTTGGCAGCCGGCTGGTCGTTGTTGTGAGC
AGCCTGTCTGTTTTTCAGGAGTGCTTCACCAAGAACGATATTGTTTTG
GCGGATCGTCCGCGTTTCCTGTCTGGCAAATACATTTTTTACAATTATA
CGACCGTAGGTTCTAGCAGCTATGGCGAACATTGGCGTAACTTGCGC
CGTATCACCAGCCTGGATGTTCTTTCCACCCACCGCATTAATAGCTTC
AGCGGTGTTCGTCGTGACGAAACGCAGCGTTTGATCCAGAAACTGGC
GGAGGAGTCCTCGACCGACTTCGCCGAAATTGAATTGACTTCGAAGT
TTTATGATATGACCTTCAACAACATCATGCGCATGATCTCCGGCAAAC
GTTATTACGGCGACGACTGTGATATGATGGATATGGAAGAGGCGAAAC
AGTTTCGTGCTATGGTGTCAGAACTCTTGCAACTGAGCGGTGCAAAT
AACAAGACCGACTTCATGCCGGTCCTGCGTCTGGTGGATTTCGAAAA
CCTGGAAAAGCGCCTGAAGAAGATCAGCAGCAAAACCGACACCTTT
CTGAGAGGCCTGATCCAAGAGCACCGCAACAAGAAGCAACACACCA
ATACCATGGTTGACCACCTGCTGTCCCTGCAAGAGTCTCAGCCGGAG
TACTACACTGACCAGATTATCAAAGGTTTAGCGTTAGGTATGCTGTTG
GCGGGCACCGACAGCTCCGCAGTCACCTTGGAGTGGGCACTGAGCT
GCCTGCTGAATCATCCGGAGGCGCTTAAGAAGGCGCGTGACGAGCTG
GAAACGCACGTGGGCCAGGATCGTCTCTTAGAGGAGTCCGACCTGAC
GAAACTGCCATATCTGAAGAACATTATTTACGAAACGCTGCGTTTATAT
ACTCCGGCACCGCTGTTGCTGCCGCATTCTTCATCCGATGAATGTATT
ATTGGTGGTTTTAAGGTGCCGCGTGAAACCATCATCCTGATCAACGCG
TGGAGCATTCATCGTGATCCGCAGATCTGGTCGGAAGCTACCAGTTTT
AAACCGGAACGTTTCGAGAAGGAGGGTGAACTGGACAAGCTGATCG
CATTTGGTCTCGGTAGACGTGCCTGCCCGGGCGAGGCTTTGGCCCTG
CGCGGTATTAGCCTGACGCTGGGCCTGTTGATTCAATGTTTTGAGTGG
AAACTGGTTGGTGACAAAGAAATCGATATGCGCGAAGAGAGTGGCTT
TACCCTGAGCCGTCTGATCCCGCTGAAAGCTATGTGCAAAGCTCGCC
CGGTTGCGAACAAACTGGTAAATACCCAGGCGGTGTCCCTCGGGGATTAA
>SEQ ID NO: 19 (GgIFR)
ATGAACATGATTGAAGTCGGTGGTTACCCAGGTAACCCAATTAACACC
AAACAATCTAATGCCCAACAACACAAGCCAACCGCTAGCTTGACTAC
TTACCAAAACAAGAAGAAAAAGAACACTCATCCATCCTTCTTTAACT
CTCCATACTTCTACTTCAACTTCAACATCCCAATGGCTGCTGAAAACA
AGATCCTGATCTTAGGTCCAACTGGTGCCATTGGACGTCATATCGTTT
GGGCTTCCGTTAAAGCCGGTAACCCAACCTTTGCTTTGGTTAGAAAG
ACCAACGGTCCAGTTAACAAACCAAAGTTGATCACTGCTGCCAATCC
AGAATCCAAGGAACAATTACTAGAATCTTACCAAAACGCTGGTGTCA
CTTTGTTGGAAGGTGACATTAATGACCACGAATCTTTGGTTAAGGCTA
TCAAGCAAGTCGATGTCGTCATCTGTGCTACCGGTAGATTATTGATCG
ATGACCAAGTTAAGATCATTGCAGCCATCAAGGAAGCCGGTAACGTT
AAGAGATTCTTCCCATCTGAATTCGGCTTGGACGTTGACAGACACGA
CTCTGTTGAACCTGTCCGTGAAGTCTTCGAAGATAAGGCTAGAATTAG
AAGAGTTATCGAAGCTGAAGGTGTTCCATACACTTACTTATGTTGTCA
CGCTTTCACCGGTTACTTTTTGAGAAACTTGGCTCAATTGGATGCTAC
TGTTCCTCCAAGAGACAAGGTTGTTATTTTGGGTGATGGTAACGTCAA
GGGTGCTTACGTTACTGAAGCTGACGTTGGTACATATACTATCAGAGC
TGCTAACGATCCAAGAACCTTGAACAAGGCTGTTCACATTAGATTGC
CAGCTAACTATTTGACCGCCAACGAAGTTGTCTCCTTGTGGGAAAAG
AAGATTGGTAAGACCTTGGAAAAGACCTACGTCCCAGAGGAAAAAG
TCTTGAAGGACATCCAAGAATCCTCTTTCCCACACAACTACTTGTTGG
CCCTATACCACTCTCAACAAATTAAGGGTGACGCTGTCTACGAAATAG
ACCCAGCTAAGGATGTCGAAGCTTACGACTTGTACTCAGATGTAAAGTACACCACTGCTG
ATGAATACTTGAACCAATTCGTCTGA
>SEQ ID NO: 20 (GgVR)
ATGGCCGAGGGCAAGGGCCGGGTATGTGTCACGGGCGGCACCGGCT
TCCTGGGATCGTGGCTGATCAAGCGTTTGCTCGAGGACGGCTACGGC
GTCAACACCACGATCCGGAGTGACCCGGAGCGCAAGCGGGACATCA
GCTTCCTCACCAACCTGCCCGGGGCGAGCGAGCGGCTGAAGATCTTC
AACGCCGACCTGTCCGACCCCGAATCCTTCGGCCCCGCGATTGAAGG
CTGCGTGGGTGTCTTCCACACCGCCTCCCCGATCGACTTCGCCGTCTC
GGAGCCCGAGGAGATCGTGACCAAGCGCACCGTCGACGGGACGCTG
GGCATCCTCAAGGCCTGCAAGAACTCCAAGACCGTCAAGCGGGTCG
TGTACACGTCCAGCGGCGCCGCGGTCAGCTGGGGGGGGACCGAGAA
GGACGTGCTCGACGAGAGCGACTGGTCGGATGTGGACATGCTGCGCT
CGGTGAAGCCGTACTCCTGGTCCTACGCCGTGAGCAAGACGATGACC
GAAAAGGCGGTGCTGGAGTTCGGCGAGCAGCACGGCCTGGACGTCG
TCACCCTGATCCTGCCGTTCATCGTCGGCCGCTTCATCTGCCCCAAGA
TCCCCGACTCCGTCGAGAAGGCGCTGGTCCTGGTGCTCGGCAAGCGC
GAGCAGATCGGCGTGACCCGGTTCCACATGGTCCACGTCGAGGACGT
GGCCAGGGCCTACATCTTCCTGCTGGAGCACCCGAACCCGAAGGGGC
GCTACAACTGCTCCCCCTTCATCGTGCCGATCGAGGAGGTCTCGGAG
CTCCTCTCCGCGAAGTACCCAGAGTACCAGATCCCGACGGTGGAGGA
ACTCAAGGAGATCAAGGGTGCCAAGCTCCCGGACCTCAAATCGAAG
AAGCTGGTCGACGCGGGGTTCGAGTTCAAGTACACCATCGAGGACAT
GTTCGACGACGCCATCCAGTGCTGCAAGGAGAAGGGATACCTGTAA
>SEQ ID NO: 21 (GgPT)
ATGTCTTCTTTATTCGGGTCAAATGTATTTGAGAGTATATCAACATATC
GTAGCTATGCTCCAAAAGCCAGTCAACATAAAAGGAAGATTCAGAAA
GAATATAATTTCCTTCGCTCCCGACAGCCTTCATTAAAGCACATTTACA
AGGGAATTGAGGGTGGAAGTACACAACAAGAGTGCAATAGGAAATAT
GTTGTAAAAACAGTGCCCAAGCCAAGCTTTGAATCAGATCCTAGGTC
TATAGATCAAAAGGACATCTTGGAATGCATTAAGAATTTTCTTGATGCT
TTATATATGTTTACTACTCCTTATTCTTTTTTCTCCTCAGCGTTGTACATT
ATCTCCGTTTCTTTCATCGTTGTGGAGAAGCTTTCCGACATAAGCCCG
CTGTTCTTCACTGGTGTGCTCCAAGCAGTTGTTCCAAATCTTTTTGTT
CACATATACATGACAGGACTAAACCAGCTTTGTGATGTTGAAATTGAC
AAGATCAACAAACCCTACCTTCCCCTGGTCTCAGGAAAAATTAGTTTT
GCAACCGGGGTTATTGTTGTCGCGTCATGCCTGTTCCTATCGCTTTGG
CTTGGATGGATTGTGGGCTCTTGGCCATCAACTTGTGTCGTGATATCT
ATCGCTATGATTTGGACTGCTTACTCTATCAATGTACCTCTCCTCAGAT
GGAAGAAGTATCCAGTCCTGGCAGCAATGTGTATATTTTTATCTCTTGC
TGTAGTGAACCCTATTGGTTATTTCTTGCATATGCAAACATTTGTGTTC
AAGAGACCTGCTTCATTTTCAAGGCCGCTCATTTTCGTGATTGCTTTC
AAAAGTTTTTTTAGTTTGGCAGTGGCATTATTTAAGGATGTTCCTGATA
TTGAAGGTGATCAGACCTTTGGCGTTCAGTCGTTTGTTGCCAGATTTG
GAAAGAAAAGAGTTTTCTGGATATGTATTTCTCTCATCGAAATGGCAT
ATGGTTTTGCTTTGTTAATGGGGGCTACTTCCGCTTTTCTATGGTCCAA
AATAGTTACAGTATTGGGCCATGCTGTTCTTGCCAGTTTTGTACTATAT
AGAGCAAAAAGCATTGATTTGCGTAGCAAAACGAGCATTACCTCTTT
TTACATGTTGATATGGAAACTGATGTATATGGAGTATTTTTTGATGCCATTGGTCCGGTGA
>SEQ ID NO: 22 (GgPTR)
ATGGCTGAAACAACTACCTCAAAATCAAAGATTCTTTTCATTGGAGG
AACTGGCTACATTGGGAAATTTATTGTTGAAGCTTCTGTTAATGCTGG
TCATCCAACTTTTGTGTTAATAAGAGATTCAACTCTGAGCAATCCAGC
GAAGTCTCCCATAATTGATAAATTCAAGAGTCTCGCAGTGAACCTCGT
TTTTGGGGACTTGTATGATCACCAGAGTCTTGTGAAAGCAATCAAAC
AAGTTGATGTTGTGATCTCCACCGTTGGACATCTTCAGCTTGGAGATC
AAGATAAGATAATATCAGCCATTAAAGAGTCTGGTAATGTTAAGAGGT
TTTTCCCTTCTGAATTTGGGAATGATGTCGACCGTACACATGCTGTAG
AGCCCGCTAAGTCTGCCTTTGCAACCAAGGCAAAAATCCGCAGGACA
ATAGAGGCCGAGGGAATTCCATACACTTACGTATCGTCCAACTTCTTT
GCTGGATATTTCTTGCCTACTCTATCTCAACCTGGTGCAACTGCCGCTC
CAAGAGACAAGGTTATAATCTTAGGGGATGGAAACCCTAAAGCTGTC
GCTAACAAGGAGGAAGATATTGCTACATATACAATCAAGGCGGTGGAT
GATCCAAGAACATTGAACAAAATACTTTATATTCGGCCGCCGGCAAAT
ACTTTATCTTTCAATGAATTGGTGAGTCTGTGGGAAAAGAAAATTGGT
AAAACTCTTGGCCGAGTTTATGTGCCAGAAGAGCAGCTCTTGAAGCA
GATTCAAGAATCGAGTCCTCCAATTAATGTCATACTAAGCATCGGCCA
CTCAGTATATGTTAAAGGTGATCATACAAATTTTGAGATTGAGCCTGC
ATTTGGTGTCGAAGCATCTGCACTGTATCCTGACGTAAAGTACACGACGGTTGACGAATATT
TGAATCAATTTGTTTGA
>SEQ ID NO: 23 (GgOC)
ATGGCTTTTACGTACAGTACCAAGAAACTTGCACTGCTCTCAATCATT
ACTATTTTCATCTCCAGCTTTCCTGAACCATCTACATCGGTTGACCTTG
AGTCAGGCTTCCTTCAATGTTTTAGTTCAGGATTGGGCAACAGTAACA
GCACAGCGGAGTTGGTGCTAACAAAAAATTCTTCCTCTTATGCTAGCC
TGCTTCAGTCTAGTATTAGAAACTTGCGGTTCATGGGAACATCTGTAC
CGAAGCCTACATTGATAGTGACTCCTAGAGATCTCTTTCATATCCAAA
CAAGCATCAAATGCTCAGTTAAACAAGGATTACAGATCCGTGTAAGG
TCCGGGGGACATGATTACGAAGGACTCTCCTACGTTTCTAATGATGAT
GTGCCATTTTTGATAATTGATCTTACTAATCTGCGAAGTATTACCATTG
ACATTAAGGATGAAACCGCTTGGGTCCAAAGTGGAGCCACTCTTGGA
GAGCTATATTATGCAATTGCTAAGAAGTCGAATGTTCACGGCTTCCCT
GCTGGTTCATGCCCTACGGTAGGGGTTGGGGGCCATTTTTCAGGTGGT
GGATTTGGAACTATATTTAGAAAGTACGGGCTGGCCGCTGATAATGTG
ATTGATGCTCAAATGGTGGATGTTAATGGAAAGATACTTAAGAATAGG
ACATTGATGGGTGAAGATTTGTTTTGGGCAATACGGGGGGGTGGTGG
CTCATCATTTGGGGTCGTTACTGCTTGGAAAGTTAAGTTAGTTCATGT
TCCTCCAAAAGTAACTGTCTTCAATATCCCCAAAACACTAGATCAAAA
TGCAAGCACCCTTTTCCACAAATGGCAAATTGTTGCAGATAAATTGCC
AGGAGAACTATTCTTGCATTCCGTGATGGGCGTCTCTAATGCCAATTC
TATTTCTTCTTCCTCGGGTGGAGGAAGAACAGTGCTTGTCAGCTTTAC
TGGATTATATCTGGGAACTGTTGAAAATCTCTTACCTCTAATGCAGAA
CAATTTTGCTGAATTGGGTCTTCAACACAATAACTGTACAGAAATGAG
TTGGATTCAGAGCGTACTTTTCTTTGCAGGATATGATCCAATCAACGA
TTCTTTAGAAGTACTCCTGCAGAGGAACCAGACATTTGGTTCTTTCAA
AGCAAAAAGCGACTATGTGATGGAGCCCATACCAACTTCAGGGTTGG
AGGGCCTTTGGAATATGTTATTGGAAGAGAATAGTAGTCCAACTTTGA
TTCTCACGCCATATGGTGGTAGGATGTCTGAGATAAGTGAGTCTGAAA
CTCCATTTCCTCATCGTAATGGTTCAATTTATGGTATACAGTATCTTGTT
TATTGGGATTCATCTGAGGAAACTCCCAAGCATATTGCATGGATGAGG
AGAGTTTATTCTTACATGGTACTTTATGTATCAAAGTTTCCGAGGGCGG
CTTATTTGAATTACAGAGACTTAGACATCGGTGTTAACCGAGGGAATA
CCTCCTACGACTATGAAGAAGCCAAATCGTGGGGTCTGAAATACTTC
AAATGTAACTTTGAGAGATTGGCTAAGGTGAAGGCAGAGGTTGATCC
TTCAAACTTCTTCCGCCATGAACAATCCATTCCTCTCCTGTTTTGA
>SEQ ID NO: 24 (GgDMT)
ATGGAGCATTTCTACATGTCTCTACTACTTTTGTTTGTAACACTGGTAA
GCCTTTCTCTCTTTTTCTTGATTTTCTATCATAACAAGCACAATATGAAT
AATAACAACAATCTACCCCCTGGGAAGATGGGTTATCCTGTTATTGGA
GAATCTCTTGAGTTTTTGTCAATGGGATGGAAGGGGCATCCTGAGAA
GTTCATATTTGACAGGATGGTTCGTTACAGTAGTGAGTTGATCAAAAC
ATCAATCTTAGGAGTCCCAACTGTGATCTTTTGTGGACCAGCTTGTAA
TAAGTTCCTTTTCAGTAATGAGAACAAATTGGTCACTGCTTGGTGGCC
AGATTCTGTAAATAAGATATTTCCCACGACTTCAAATAGCAAAGAGGA
ATCCAAGAAAATGCGGAAATTGCTTCCTCAATTTCTTAAGCCTGAAGC
TTTACAGAGGTATGTTGGCATAATGGATACTCTGGCTCAGCGACATTT
TGCTAGTTTGTGGGAGGAGAAAACTCATGTTACTGTTTACCCGCTGGC
AAAACGGTACACATTCATGCTTGCTTGCCGCCTATTCATGTCTGTTGA
AGATGAGAATCATGTGGCGAAGTTTCGCGAACCTTTCCACTTGCTTGC
ATCTGGTATCATATCAGTCCCAATTGATCTACCATGGACACCATTTAAC
AGAGGCATCAAGGCATCAAATTTTATTAGGAAGGAACTGTTAAAGAT
AATTAGACAGAGAAAAGTAGATTTGGCCCAGGGCGTAGCGTCCCCTA
CTCAAGATATTTTAAGCCATATGTTGTTAACATGTGATGATGAAAACG
GTGAATTTATGACAGAATTGAATATTGCTGATAAAATACTCGGTCTCCT
CATTGGTGGTCATGACACAGCATCTGCCGCATGCACTTTCATCGTGAA
GTATTTGGCTGAACTTCCACACATTTATGATAGAGTTTATCAAGAACA
AATGGAAATAGCTAATTCAAAATCTCCAGGAGAGCTGCTTAATTGGGA
TGACATTAACAAGATGCGTTATTCATGGAACGTTGCTAGCGAAGTCAT
GAGGGTGGCCCCTCCTCTTCAAGGGGGTTTTCGAGAAGCAATTAATG
ATTTTGTGTTCAATGGATTTTCGATTCCCAAGGGTTGGAAACTCTATTG
GAGTGCAAACTCCACGCATAAAAATCCCGAGTATTTTCCAGCCCCGG
AGAAATTCGATCCAACCAGGTTTGAAGGAAATGGCCCTGCTGCATAC
ACCTTTGTTCCATTTGGAGGAGGGCCAAGAATGTGTCCTGGAAAAGA
ATATGCTAGATTAGAGATATTGGTCTTTATGCATAATCTTGTTAAGAGAT
TCAAATGGGAAATGCTAATTCCAGAAGAAAAAATCGTTGTGGACCCG
TTACCTATGCCAGCAAATGACCTGCCCATTCGTCTTTACCCTCACAACACCTGA
>SEQ ID NO: 25 (P1OMT9)
ATGGGTTCCAACAACGGTCGTAAAGCTTCTGAAATTTTCCAAGGTCA
AGCTTTGCTATACAGACACATGTTTGCTTTCGTCGACTCCATGTGTTT
GAAGTCCATTGTCGAATTAGGTATCCCAAACATCATTCACAAGCATGG
TCAACCAATCACCTTATCCGAATTGATGTCCATCTTGCAAGTCCCTCC
AGCTAAGGTTGGTCACGTTCAATCTTTGATGAGATATCTGGCTCACCA
CGGTTTCTTCGAAAGATTGAGAATTCACGAAAAGGATGCTTACGCTC
TGACTGCTGCTTCCGAATTGTTAGTCAAGGGTACCGAACCATACTTGG
CTCCAATGGTTGAATGTATGTTGGATCCAACTTTGTCTGCTTCTTTCCA
CCAAATGAAGAAGTGGGTTTACGAAGAAGATTTGTCAGTTTTCGACA
TCTCCTTGGGTTCTAATTTATGGGATTTCTTGACCAAGAACCCAGCCT
ACAACGAAATCTTCAACGAAGCTATGGCTTCTGATTCTCAAATGTCTA
ACTTGGCCTTGAGAGATTGCAAGTTAGTCTTTGAAGGTTTGGAATCC
ATCGTGGATGTCGGTGGTGGTACTGGTACTACAGCCAAAATTATTTGT
GAAGCCTTCCCAAACTTGAAGTGTATCGTCTTTGACAGACCACAAGT
TGTTGAAAACTTGTTAGAGAACAACAACTTGACCTACGTTGGTGGTG
ACATGTTCAAGTCTATTCCAAAGGCCAACGCTGTCTTATTGAAATGGA
TTCTACATGACTGGACTGACAAGGACTGTCGTAAGATTTTGGAAAAC
TGTAAGGAAGCTATCTCTAACAATAGTAAGAGAGGTAAGATCATCATC
ATTGACATTGTTATCAACGAAAAGCAAGACGAGCACAAGGTCACTGA
ATTGAAATTGTTGTTCGATGTTGCTATGGCCTGTGTTTTGAACGGTAA
GGAAAGAAACGAAGAAGAATGGAAGAAGCTTTTCATGGAAGTTGGA
TTGCAAGACTACAAAATTTCTCCATTGACCGGTTACTTGTCTTTGATAGAAATCTACCCAT
GA
>SEQ ID NO: 26 (GmCYP81E1)
ATGGAAATTTTGTCTTTGTTGTCCTACTCAGTTTTCTATTTGGCTTTGT
TCTTCATCTTCAACATCGTTATCAGAGCTAGAAAGTTCAAGAATTTGC
CTCCAGGTCCACCATCTTTGCCAATTATCGGTAACCTCCACCACTTAA
AGAGACCATTGCATAGAACCTTCAAGGGTTTGAGTGAAAAGTACGGT
CACGTTTTCTCCTTGTGGTTCGGTTCCAGACTGGTCGTCGTTGTCTCC
TCCGCCTCTGAATTCCAACAATGTTTCACCAAGAACGATGTTGTTCTA
GCCAACAGACCAAGATTCCTAAGCGGTAAATACATTTTCTACAACTAC
ACCACTTTGGGTTCTACCTCTTACGGCGAACATTGGAGAAACTTACGT
AGAATCACTGCTTTGGATGTCTTGTCCAACCATAGAATTAACTCTTTC
TCTGGTATCAGAAGAGACGAAACTCAAAGATTGATCACCAGATTAGC
TGATGACTCTTCCACCAACTTCGCTGAAATGGAATTGTCTTCTAGATT
GTACGACATGACTTTCAACAATATCATGAGAATGATCTCCGGTAAGAG
ATACTATGGCGAAGACTGTGATACTTCTGACTTGCAAGAAGCTTCTCA
ATTCAGAGACATGGTTTCCGAATTGTTGCAATTGTCTGGTGCTAACAA
CAAAACTGATTTCATGCCATTGTTGAGATTCTTGGACTTCGAAAACTT
GGAAAAAAGATTGAAGGACATTTCTGGTAAGACCGATGCTTTTTTGA
GAGGATTGATTGAAGAACACAGAACTAAGAAGGAAAGAGCCAACAC
CATGATTGACCACTTGTTAAATTTACAAGATTCCCAACCAGAATACTA
CACCGACCAAATCATCAAGGGTTTAGCCTTGGCCATGTTGTTGGCTGG
TACTGACTCCTCTGCTGTCACTTTGGAATGGTCCATGTCTAACTTGTT
GAACCACCCAGAAGTTTTGAAGAAAGTTAAGGATGAATTGGACACTC
ACGTCGGTCAAGATCGTTTAGTCGACGAATCTGATTTGCCAAAGTTG
ACTTACTTGAAGAACGTTATCAACGAAACCTTAAGATTATACACTCCA
GCTCCATTGTTGCTGCCACACTCTACTTCCGATGAATGTAACATTGGT
GGTTACAAGGTTCCTCAAGACACCATTGTTTTGATCAACGCTTGGGCT
ATTCACCGTGATCCAGAATTATGGACTGAAGCTACCACATTTAAGCCA
GAACGTTTTGAAAAGAAAGGTGAATTGGAAAAGTTGATTGCTTTCGG
TATGGGTAGAAGAGCTTGTCCAGGTGAAGGTCTAGCCATTCGTGCAA
TCTCTATGACCTTGGCTTTGTTGATTCAATGCTTTGACTGGAAGTTAAT
CAACGGTGACAAGATCGACTTGGCTGAGAGAGATGGTTTCACTTTGA
CCAAATTGGTCCCATTGAAGGCTATGTGTAAGTCTCGTCCAGTTATTA
ACAAGGTCTTTAAGCAATGA
>SEQ ID NO: 27 (MsIFR)
ATGGCTACCGAAAACAAGATCTTGATTTTGGGTCCAACTGGTGCCATC
GGTAGACACATTGTCTGGGCTTCCATCAAGGCTGGTAACCCTACCTAC
GCCTTAGTCAGAAAGACCCCAGGTAATGTTAACAAGCCAAAGTTGAT
CACTGCTGCTAACCCAGAAACCAAAGAAGAATTGATTGACAACTACC
AATCTTTGGGTGTCATCTTGTTGGAAGGTGACATCAACGACCACGAA
ACATTGGTCAAGGCTATTAAGCAAGTCGATATTGTTATCTGTGCTGCC
GGCCGTTTATTGATCGAAGACCAAGTCAAGATAATCAAAGCTATCAAG
GAAGCAGGTAACGTCAAGAAGTTCTTCCCATCTGAATTCGGTTTAGA
CGTTGACAGACATGATGCTGTTGAACCAGTTAGACAAGTTTTTGAAG
AAAAAGCTTCCATTAGAAGAGTTATTGAAGCTGAAGGTGTTCCATAC
ACTTATTTGTGTTGTCACGCTTTCACCGGTTACTTCTTGAGAAACTTG
GCTCAATTGGACGCCACCGACCCTCCAAGAGACAAGGTTGTTATTTT
AGGTGATGGTAACGTTAAGGGTGCTTACGTTACTGAAGCTGATGTTG
GTACTTTTACTATCCGTGCTGCTAATGACCCAAACACTTTAAACAAAG
CCGTCCATATCAGATTGCCAAAGAACTACTTGACTCAAAACGAAGTC
ATTGCTTTGTGGGAAAAGAAGATTGGTAAGACCTTGGAAAAGACTTA
CGTCTCAGAAGAACAAGTTTTGAAGGATATCCAAGAATCTTCTTTCCC
ACACAACTATCTACTGGCTTTGTACCACTCTCAACAAATCAAGGGTGA
TGCTGTATACGAAATTGATCCAGCTAAGGACATTGAAGCCTCCGAGGC
TTACCCAGACGTCACCTACACCACTGCCGATGAATACTTGAACCAATTCGTCTGA
>SEQ ID NO: 28 (MsVR)
ATGGCCGAAGGTAAAGGTAGAGTCTGTGTCACCGGTGGTACTGGCTT
CTTGGGTTCCTGGATTATAAAGTCTTTATTAGAAAACGGTTACTCCGTC
AACACCACTATCAGAGCAGATCCAGAGAGAAAGAGAGATGTCAGTTT
CTTGACTAATTTGCCAGGTGCCTCTGAAAAATTGCACTTCTTCAACGC
CGACTTGTCTAACCCAGATTCTTTTGCTGCTGCTATTGAAGGTTGTGT
TGGTATCTTCCACACTGCTAGCCCAATCGACTTTGCTGTTTCCGAACC
AGAAGAAATCGTTACCAAGAGAACCGTTGACGGTGCTCTTGGAATCT
TGAAAGCTTGTGTTAACTCTAAGACTGTCAAGCGTTTCATTTACACCT
CTTCTGGTTCTGCTGTTTCTTTCAATGGTAAGGACAAGGATGTTTTGG
ACGAATCCGACTGGTCTGACGTCGACTTGTTGAGATCTGTCAAGCCA
TTCGGTTGGAACTACGCTGTCTCCAAGACCCTAGCTGAAAAGGCTGT
ATTGGAATTCGGTGAACAAAACGGTATTGACGTTGTTACTTTGATTTT
GCCTTTCATTGTCGGTCGTTTCGTCTGCCCTAAGTTGCCAGACTCCAT
TGAAAAGGCTTTGGTCCTAGTTTTGGGTAAGAAAGAACAAATTGGTG
TTACCAGATTTCACATGGTTCACGTTGATGACGTCGCCAGAGCCCATA
TCTATTTATTGGAAAACTCCGTTCCAGGTGGTAGATACAACTGTTCTC
CATTCATCGTTCCAATTGAAGAAATGTCACAATTGTTGTCCGCTAAGT
ACCCAGAATACCAAATCTTGACTGTGGATGAATTGAAGGAAATCAAG
GGTGCTAGATTACCAGATTTGAACACTAAGAAGTTAGTCGATGCTGGT
TTCGACTTCAAGTACACAATCGAAGACATGTTCGATGATGCTATCCAATGTTGTAAGGAAAA
GGGTTACTTGTGA
>SEQ ID NO: 29 (tPcM4DT)
GCATCTCAACACAAGAAGAAGACTCAAATCGAATACAACGTCTTGAG
ATTCCAAAGACCATCCTTGGGTCACGGATACAACTTCTCCGGTGGTG
GTGGCTCCACTTACCAAGAATGTAACAGAAAGTACGCCGTTAAGGCT
GTCTACGACCAACCAAAGGACTTCGAATTGGAAGCTTCCAACCCAAA
GAACATTTTGGACTCTGCCAAGAAATTCTTGGCTGCTTTTTACTACTT
CAGTTACCCATACACCATGATTGGTATCACTTTGTGCGCTTTCTGTTCC
TCTGCTTTAGCTGTCGAAAAGTTGTCTGATATCTCCCTCCCTTTCTTCA
TCGGTGTCTTGCAAGCTGTTATCCCACAATTGTTTATTGAAATTTACTT
GTCTGGTGTTAACCAATTATATGACTTGGAAATCGACAAGATTAACAA
GCCACATTTGCCAATGGCTTCTGGTCAATTCTCTTTCAAGACCGGTGT
CATCATGTCTGCCGCCTTTTTGGCTTTGTCCTTCGGTTTCACCTGGTTG
ACCGGATCTTGGCCATTGATCTGGAACTTGGTTGTTATTGCCTCCTCCT
GGACTGCTTACTCTATTGACGTTCCATTCATGAGATGGAAGAGATACC
CATTAGTTGCTGCTATGTGTATGATCGCTACCTGGGGTCTGGCTTTACC
AATCTCTTTTTTCCACCACATGCAAACTTTCGTTTTGCAACGTCCTATT
GGTTTCCCAAGATCTTTGGGTTTCTTGGTTGCTTTCATGACTTTTTATT
CCATGGGTATTGCTTTGAGCAAGGATATTCCAGATGTTGAAGGTGACA
AGGAACATGGTATCAATTCTTTCTCAGTCTTGTTGGGTCAAAAGCGTA
TCTTCTGGATCTGTGTCTCATTGTTTGAAATGGCTTTCGGTGTTGGTCT
ATTAGGTGGTGTTACTTCTCCACACTTCTGGACCAAGATCATTACCGG
TCTAGGTAACGTCATTTTGGGTTCCATCTTGTGGTACCAAGCTAAGTC
TTTCGATTTGACAGATAAAGCTTCTACTGGTTCTTTCTACATGTACATC
TGGAAATTGTTGTACGCTGCCTTATTGTTGATGGCCTTCGTCAGATGA
>SEQ ID NO: 30 (GgPTR)
ATGGCCGAAACCACCACTTCTAAGTCTAAAATATTGTTTATTGGTGGT
ACCGGTTACATTGGCAAGTTCATTGTTGAAGCCAGTGTCAATGCCGGT
CACCCAACTTTCGTCTTGATTAGAGATAGCACTTTGTCTAACCCAGCG
AAGTCCCCAATCATTGATAAATTCAAGTCTTTGGCTGTCAACTTGGTT
TTCGGTGACTTGTACGACCACCAATCTTTGGTCAAGGCCATCAAACA
AGTTGATGTAGTGATCTCCACAGTCGGTCATTTGCAATTGGGTGACCA
AGACAAGATTATTTCCGCTATCAAGGAATCCGGTAATGTCAAGAGATT
CTTCCCATCAGAATTCGGTAACGACGTTGACAGAACCCACGCTGTCG
AACCAGCTAAATCCGCTTTCGCTACCAAGGCTAAGATCAGACGTACTA
TCGAAGCTGAAGGTATCCCATACACTTACGTTTCTTCCAACTTTTTTG
CTGGTTACTTCTTGCCAACCTTATCCCAACCAGGTGCTACTGCTGCTC
CAAGAGACAAGGTTATCATCTTGGGTGACGGTAACCCAAAGGCTGTT
GCTAACAAGGAAGAAGATATTGCTACTTACACTATCAAGGCAGTCGAT
GACCCACGTACCTTAAACAAGATTTTGTACATCAGACCACCAGCCAA
CACTCTATCTTTCAACGAATTGGTTTCCTTATGGGAAAAGAAGATCGG
TAAGACCTTGGGTAGAGTCTATGTCCCAGAAGAACAACTGTTGAAGC
AAATTCAAGAGTCTTCTCCACCTATCAACGTTATCTTATCTATTGGTCA
CTCTGTTTACGTCAAGGGTGACCACACCAACTTCGAAATTGAACCTG
CTTTCGGTGTTGAAGCCTCTGCTTTGTATCCAGATGTTAAGTACACCA
CTGTTGATGAATACTTGAACCAATTCGTTTGA
>SEQ ID NO: 31 (GgOC)
ATGGCTTTCACTTACTCCACTAAGAAGTTGGCTTTGTTGTCTATCATCA
CTATATTCATCTCCTCCTTCCCAGAACCATCTACTTCTGTTGATTTGGA
ATCTGGTTTCTTGCAATGTTTCTCTTCTGGTCTAGGTAACTCTAACTCA
ACTGCTGAATTGGTCTTGACCAAGAACTCTTCCTCTTACGCCTCCCTG
TTGCAATCTTCCATTCGTAATCTGAGATTCATGGGTACCTCGGTCCCA
AAGCCAACCTTGATTGTCACTCCACGGGATTTGTTCCATATTCAAACC
TCTATTAAGTGTTCTGTTAAGCAAGGTTTGCAAATCAGAGTTAGATCA
GGTGGTCACGACTACGAAGGTTTGTCCTACGTCTCCAACGATGACGT
TCCATTCCTGATTATCGACTTGACCAACTTGCGTAGTATCACCATTGAC
ATCAAGGACGAAACTGCTTGGGTTCAATCTGGTGCAACTTTGGGTGA
ATTGTACTACGCTATTGCCAAGAAGTCTAACGTCCACGGTTTCCCAGC
TGGTTCTTGTCCAACCGTCGGTGTTGGTGGTCACTTCTCTGGTGGTGG
TTTCGGTACCATCTTCAGAAAGTACGGTCTAGCCGCTGATAACGTTAT
CGACGCTCAAATGGTTGATGTCAACGGTAAGATCTTGAAGAACAGAA
CCTTGATGGGTGAAGACTTGTTCTGGGCTATCAGAGGCGGTGGTGGT
TCTTCCTTTGGTGTCGTCACCGCTTGGAAGGTTAAATTGGTCCATGTT
CCACCAAAAGTGACAGTTTTCAACATCCCTAAGACCTTGGACCAAAA
CGCTTCTACCTTGTTCCACAAGTGGCAAATCGTTGCTGACAAGTTACC
AGGTGAATTGTTTTTGCACAGTGTCATGGGTGTTTCCAACGCCAACTC
CATTTCCTCATCTTCCGGTGGCGGTCGTACTGTCTTAGTTTCTTTCACC
GGCTTGTACTTGGGTACTGTTGAGAACCTATTGCCATTGATGCAAAAC
AACTTTGCTGAATTAGGTTTACAACACAACAACTGTACTGAAATGTCC
TGGATTCAATCCGTTTTATTCTTTGCCGGTTACGACCCAATCAACGAC
TCTTTGGAAGTTTTGTTACAAAGAAACCAAACTTTCGGTTCCTTCAA
GGCCAAGTCTGACTACGTTATGGAACCTATTCCAACTAGCGGGTTGG
AAGGTTTGTGGAACATGTTGCTCGAAGAAAATAGCTCTCCAACTTTG
ATCTTGACTCCATACGGTGGTAGAATGTCTGAAATTTCTGAATCTGAA
ACCCCGTTCCCACACAGAAATGGTTCCATCTACGGTATCCAATACTTA
GTTTACTGGGATTCCTCCGAAGAAACCCCAAAGCATATTGCTTGGATG
AGAAGAGTCTACTCTTACATGGTTTTGTACGTTTCTAAGTTCCCAAGA
GCTGCTTACTTGAACTACAGAGATTTGGATATTGGTGTTAACAGAGGT
AACACTTCCTATGACTATGAAGAAGCCAAATCTTGGGGTTTAAAGTAT
TTCAAATGCAATTTCGAAAGATTGGCTAAAGTCAAGGCTGAAGTCGA
TCCTTCTAACTTCTTCAGACACGAACAATCTATTCCATTGTTATTTTGA
>SEQ ID NO: 32 (NhDMT)
ATGCTCGTTGACACTGGTTTAGGATTGATTTCTGAATTGCAAGCCAAG
TTGGGTTGGGCTGTTTTGTTGCAAATTGTCCCGATCACCATCGTCGCT
TACAACTTACTGTGGTTCATCTACGCTTCCTTCTTTTCCTCTTTGAGAA
AGATTCCAGGTCCATTCTTGGCTCGTATCTCCCGTGTCTGGGAAATGA
AGAAGACTGCTACCGGTAACATTCACGAAATCATGATGGACTTGCAC
AGACGTCACGGTGCTATCGTCAGAATCGGTCCAAGGAGATATGACTT
CGACACCATGGAAGCTTTGAAGATCATTTACAGAATTGGTAATGCCTT
GCCTAAAGCTGACTACTACAAGCCATTCGGTTTGCCATCTTTCCCAAA
CTTGTTTGATGAACAAAACCCAGCTAGACATTCCGCTATCAAGAAAC
AAGTTGCTTCTTTGTACACTATGACTGCTTTATTGTCTTACGAAGAAG
GTGTCGATGGTCAAACCGCCATCTTGAAGGAACAATTGCAAAGATTC
TGTGACCAAAAGCAAGTCATTGATTTGCCAAGATTCTTGCAATACTAC
GCTTTCGATGTTATCGGTGTTATCACTGTTGGTAAGTCTATGGGTATGA
TGGAATCCAACTCTGATACTAATGGTGCTTGCTCTGCTTTGGACGGTA
TGTGGCACTACGCCTCCATGATGGCTTACATCCCTAACATGCACGCTT
GGTGGTTGAGATTGAGCTCTCTTTTGCCAATCGAAGTTCCAATTAAGG
GTTTGACTGAATACGTTGAAAGAAGAATCATCCAATACAGATTAAAG
GCTGCTGAATTCGGTGATGATGCTGCCTTGAAAGGTGAAAACAACTT
TTTGGCTAAGTTGTTGTTGATGGAAAAGAAGGGTACAGTCACTCCAG
TGGAAACTCAACAAGCTGTCGGCTTGAACATTGGTGCTGGTTCTGAC
ACTACTGCGAACGCTTTGAGTACCATCTTGTATTATTTATACACCAACC
CAAGAACCCTGCACACTTTGAGAGAAGAGTTGGAACGTTACGTCAA
GGATGGTCCAATCTCTTTCCAGCAATCTCAATCCATGCCATACTTGCA
AGCTGTCATCAAGGAAGCCCTAAGATTACATCCAGGTGTTGGTACCC
AATTGACCAGAGTTGTTCCAAAGGGTGGTTTAGTCATTGAAGGTCAA
TTCTTCCCAGAAGGTACCGAAGTCGGTGTTAACGGTTGGGCTCTATAC
CACAACAAGGCCATTTTTGGTAACGATGCCTCCATCTTCAGACCAGA
AAGATGGTTAGAAGCTAACGAAAACATTAACATCGGTGGTTCTTTCG
CCTTCGGTGCCGGTTCCAGATCTTGTATCGGCAAGAATATCAGCATTT
TGGAAATGTCCAAGGCTATTCCACAAATTGTTAGAAACTTCGACATTG
AAATTAACCACGGTGACATGACCTGGAAGAACGAATGTTGGTGGTTC
GTTAAACCAGAATACAAGGCTATGATTAAGCCAAGAAGATGTTGTTTG
TCTAGAGACGAATCTCTGGTTTGA
>SEQ ID NO: 33 (GgOMT)
ATGGACTTCTCTACCAACGGTTCTGAAGAATCAGAATTATACCACGCT
CAAATCCATTTATATAAGCACGTTTACAACTTCGTTTCTTCCATGGCTC
TGAAGTCTGCTATGGAATTGGGTATTGCCGATGTTATTCACAATCACG
GTAAGCCAATTACTTTGCCACAATTGGCTAGTGCTTTGAAGTTGCACC
CATCTAAGGTCGGCGTTTTATACAGATTCTTGAGATTATTGACTCACAA
TGGTTTTTTCGCTAAGACCACTGTTCCGTCACAAAACGGTAAGGAAG
GTGAGGAAGAAGAAACCGCTTACGCTTTGACCCCACCTTCCAAATTG
TTGGTCAAGGGTAAGCCAACTTGTTTGGCCTCCATTGTCAGAGGTGC
TTTGCATCCAAGCTCTCTTGACATGTGGAGATCTTCTGAAAAGTGGTT
TAACGAAGACAAGGAATTAACATTGTTCGAATCCGCCACTGGTGAAT
CTTTCTGGGATTTCTTGAACAAGGACTCCGAATCTGGTACTTTGTCCA
TGTTCCAAGAAGCCATGGCTGCTGACTCTCAAATGTTCAAGTTGGCC
TTGAAGGAATGTAGACACGTCTTTGAAGGTTTGGAATCTTTAGTCGAT
GTTGGTGGAGGTACCGGTGGTGTCACCAAGTTGATCCACGAAGAATT
CCCACATTTGAAATGCACCGTCTTCGACCAACCACAAGTTGTTGGTA
ACTTGTCTGGTAACGAAAACCTAAAATTCGTCGGTGGTGACATGTTTA
AGTCCATCCCACCAGCTGATGCTGTCTTGTTGAAATGGGTTTTGCACG
ACTGGAACGATGAACTATCTTTGAAGATTTTGAAGAACTCCAAGGAA
GCTATCTCCGGTAAGGGTAAAGAAGGTAAGGTTATCATCATTGATATTT
CTATCGACGAAGCTTCCGGTGATCGTGAATTGACTGAATTGCAATTGG
ACTACGATTTGGTTATGTTGACCATGTTCAACGGTAAGGAAAGAGAA
AAGAAGGAATGGGAAAAATTAATCTCTGACGCTGGTTTCTCCTCTTAC
AAGATCACTCCAATCTGTGGTTTCAAGTCCTTGATTGAAGTTTTCCCATGA
>SEQ ID NO: 34 (GgI2′H)
ATGGAAATCTTGTCCTTGTTGTCTTACTCCGTTTTTTACGTTGCTTTGT
TCTTCATCTGTAACTTGTTATTCCAAGCTAGAAAGTTCAAGAATTTGC
CACCTGGTCCACCATCTCTCCCAATCATTGGTAACTTACATCATTTGAA
GAGACCATTGCACAGAACTTTCAAGGGATTGTCTGAAAAGTACGGTC
ACGTTATCTCTTTGTGGTTCGGTTCTAGATTGGTTGTCGTCGTGTCTTC
CTTATCTGTCTTCCAAGAATGCTTCACCAAGAACGATATTGTTCTCGC
TGACAGACCAAGATTCTTGAGTGGTAAATATATCTTCTACAACTACAC
CACTGTCGGTTCCTCTTCCTACGGTGAACACTGGAGAAACTTAAGAA
GAATCACTTCCTTAGATGTCTTGTCCACCCACAGAATTAACTCTTTCT
CTGGTGTTAGAAGAGATGAAACCCAAAGACTGATCCAAAAGTTGGCC
GAAGAATCATCCACTGATTTTGCTGAAATTGAATTGACCTCCAAGTTT
TACGATATGACTTTCAACAACATCATGAGAATGATTTCCGGTAAGAGA
TACTACGGCGATGACTGTGACATGATGGACATGGAAGAAGCTAAGCA
ATTCAGGGCCATGGTTTCCGAATTGCTACAATTGTCTGGTGCTAACAA
CAAGACCGACTTCATGCCAGTCTTGAGATTGGTCGATTTCGAAAACTT
GGAAAAACGTTTAAAGAAGATCTCCTCTAAGACTGACACCTTCTTGC
GTGGTTTGATCCAAGAACACAGAAATAAGAAGCAACACACCAACAC
CATGGTTGACCACTTGCTGTCTTTGCAAGAATCTCAACCAGAATACTA
TACTGACCAAATCATCAAGGGTTTAGCCTTGGGTATGTTGTTGGCTGG
TACCGATTCCTCAGCTGTTACTTTGGAATGGGCTCTATCTTGTTTGTTG
AACCACCCAGAAGCCTTGAAGAAGGCAAGAGACGAATTAGAAACCC
ATGTCGGTCAAGATCGTTTGTTGGAAGAATCCGATTTGACAAAATTGC
CATACTTGAAGAACATTATCTACGAAACTTTGAGATTGTACACTCCAG
CTCCATTATTGTTGCCTCACTCTTCTTCTGACGAGTGTATCATCGGTGG
TTTCAAGGTTCCAAGAGAAACTATTATTTTGATTAACGCTTGGTCTATT
CACCGTGACCCACAAATCTGGTCTGAAGCTACTTCTTTCAAGCCAGA
AAGATTTGAAAAGGAAGGTGAATTGGACAAATTAATTGCTTTCGGTC
TGGGTAGAAGAGCTTGTCCAGGTGAAGCTTTGGCCTTGAGAGGTATC
TCTTTGACCTTGGGTCTATTGATTCAATGTTTCGAATGGAAGTTGGTT
GGTGACAAGGAAATTGACATGCGTGAAGAATCTGGTTTCACTTTATCC
AGATTAATTCCATTGAAGGCTATGTGTAAGGCTCGTCCAGTTGCCAAC
AAATTAGTTAACACTCAAGCTGTCTCTTTGGGTGATTGA
>SEQ ID NO: 35 (GgVR)
ATGGCTGAAGGTAAGGGTAGAGTCTGTGTCACCGGTGGTACTGGTTT
CCTAGGTTCATGGTTAATCAAGCGTTTATTGGAAGACGGTTACGGCGT
CAACACTACCATCAGATCCGACCCAGAAAGAAAGAGAGACATTTCTT
TCTTGACTAACTTGCCAGGTGCCTCCGAAAGATTAAAAATCTTCAATG
CTGACTTGTCCGACCCAGAATCCTTCGGACCAGCTATTGAAGGTTGT
GTTGGTGTCTTCCACACTGCTTCCCCAATTGACTTCGCCGTCAGTGAA
CCAGAAGAAATTGTCACCAAGCGTACTGTTGATGGTACTTTGGGTATC
TTGAAGGCTTGCAAGAACTCCAAGACTGTTAAGAGAGTTGTCTACAC
CTCTTCTGGTGCTGCTGTCTCTTGGGGTGGTACCGAAAAGGATGTCTT
GGACGAATCTGACTGGTCCGATGTGGACATGTTGAGATCTGTCAAGC
CATACTCTTGGTCTTACGCTGTTTCCAAGACCATGACTGAAAAGGCCG
TCTTGGAATTCGGTGAACAACACGGTTTGGATGTTGTTACATTGATTT
TGCCATTCATTGTCGGTAGATTTATCTGTCCAAAGATCCCTGACTCTGT
TGAGAAAGCTTTGGTCCTCGTTTTGGGTAAGAGAGAACAAATCGGTG
TTACCAGATTCCACATGGTTCATGTTGAAGACGTTGCTAGAGCTTACA
TCTTTTTGCTTGAACACCCAAACCCAAAGGGGAGATACAACTGTTCT
CCATTTATTGTTCCAATCGAAGAAGTTTCTGAATTGTTGTCTGCCAAAT
ACCCAGAATACCAAATTCCAACCGTTGAAGAATTAAAAGAAATCAAG
GGTGCTAAGTTGCCTGATTTGAAGTCTAAAAAGTTAGTTGATGCTGGT
TTCGAATTCAAGTACACTATTGAAGATATGTTCGACGATGCCATCCAATGTTGTAAGGAAAA
GGGTTATTTGTGA
>SEQ ID NO: 36 (GgPT)
ATGTCTTCTCTATTTGGCTCTAACGTTTTCGAATCCATTTCCACTTACA
GATCTTACGCTCCAAAGGCTTCTCAACACAAGAGAAAGATCCAAAAG
GAATACAACTTCTTAAGATCCAGACAACCATCTTTGAAGCACATTTAC
AAGGGTATCGAAGGTGGTTCCACTCAACAAGAATGTAACAGAAAGTA
CGTCGTTAAGACCGTTCCAAAACCATCCTTTGAATCTGACCCAAGATC
CATCGACCAAAAGGATATTTTGGAATGTATCAAGAACTTTTTGGATGC
CCTTTATATGTTCACCACTCCATACTCGTTCTTTTCCTCCGCTTTGTAC
ATCATTAGTGTTTCTTTCATCGTTGTTGAAAAGTTGTCTGACATTTCCC
CATTGTTCTTCACCGGTGTCTTGCAAGCTGTGGTTCCAAACTTGTTCG
TCCATATCTACATGACAGGTTTGAACCAATTGTGTGATGTCGAAATCG
ACAAGATCAACAAGCCTTACTTGCCATTGGTCTCTGGTAAAATTTCCT
TTGCCACCGGTGTAATTGTCGTTGCTTCTTGTCTCTTCTTGTCCTTGTG
GTTGGGTTGGATCGTCGGTTCTTGGCCTTCTACCTGTGTCGTCATCTC
CATAGCCATGATCTGGACTGCTTACTCTATCAATGTTCCATTATTGAGA
TGGAAGAAATACCCAGTTTTGGCTGCTATGTGTATTTTCTTGTCTTTGG
CCGTCGTTAACCCAATTGGTTACTTCTTACACATGCAAACTTTCGTCTT
CAAGCGTCCAGCTTCCTTCTCTAGACCATTGATCTTCGTTATAGCTTTC
AAATCTTTCTTCTCTTTAGCCGTCGCTTTATTCAAGGACGTCCCAGAC
ATTGAAGGTGATCAAACCTTCGGTGTCCAATCTTTTGTTGCCCGTTTC
GGTAAGAAGAGAGTTTTCTGGATCTGCATCAGTTTGATTGAAATGGCT
TACGGTTTCGCTTTGTTGATGGGTGCTACTTCTGCTTTCTTATGGTCTA
AGATTGTCACTGTTTTGGGTCACGCTGTTTTGGCATCATTCGTTTTGTA
TAGAGCTAAGTCTATTGATTTGAGATCCAAGACTTCCATTACCTCTTTC
TACATGTTGATCTGGAAGCTAATGTACATGGAATACTTCTTGATGCCATTAGTTAGATGA
>SEQ ID NO: 37 (GgPTR)
ATGGACTCTACTGCTGCCTCCAAGATCTTGGTTATTGGTGGTACTGGT
TACATGGGTCGTTTTCTGGTCGAAGCTTCTGCCAAGGCCGGTCATCCT
ACCTTTGCTTTAGTACGTCACAGTACTGTTACCAACCCAGACAAGTCC
TCTATTATTCACTCTTTCAACACCCTAGGCGTCAATCTATTGTTGGGTG
ACATTAATGATCATCAATCTTTGGTCAAGGCCATCAAGCAAGCTGATG
TTGTCATCTCTACCGTTAACCACCAATATATTTCTGACCAATACAAGAT
TATTAGCGCTATCAAGGAAGCAGGTAACATCAAACGTTTCTTCCCATC
TGAATTCGGTAACGACGTTGTCAGAACTCACGGTGTCGACGGTGCTA
AGGCTTTGTTCGATATCAAGGCTAAATTCAGAAGAACCATCGAAGCT
GAAGGTATCCCTCACACTTACGTCGTTGCCAACTTCTTGACTCAACAC
TTCTTGCCAACTAGATCCAGATTATTGGCTATTGCTGCTCCATTAGATA
AGGTTGTCATATTGGGTGATGGTAACACCAAGGCCACATTCAACACT
GAAGAAGGTGTTGCTACCTTCACTATCAGAGCTGTCGACGACCCAAG
AACTTTGAACAAGATCTTATACATTAGACCACCAGCTAACACTTTGTC
CTACAACGATTTGGTTTCCTTGTGGGAAAAGAAGACCGGTAACACCT
TGGAAAGAGTTTATGTTCCAGAAGAACAAGTTTTGAGATTGATCCAA
GAATCTTCTTACCCATTGAACATGGCTTTGTCCATCTGTCACGCTGCTT
ACGTTAAGGGTGACCACACCAACTACGAAATTGAACCATCCTTCGGT
GTTGAAGCCTTGGAATTGTACCCAGATGTCAAGTTCACCACTGTGGAT
GACTACTTGGAACAAAACAGAGACTGTACTCCATTCTACTTAAATCA
ATTGATTCCAATCACTAACGGTACCAAATTTTGA
>SEQ ID NO: 38 (GgOC)
ATGGCTTTCACTTACTCCACCAAGAAATTGGCTTTGTTGTCTATTATCA
CCATCTTCATCTCCTCTTTCCCAGAACCATCTACCTCTGTTGACTTGGA
ATCCGGTTTCCTGCAATGTTTTTCCAGTGGTTTGGGTAACTCTAATTCC
ACTGCAGAATTGGTTTTGACCAAGAACTCGTCCTCTTACGCCTCCTTG
TTGCAATCCTCCATCCGTAACCTAAGATTCATGGGTACTTCCGTCCCA
AAGCCAACTTTGATTGTCACTCCAAGAGACTTGTTCCATATCCAAACC
TCCATCAAATGCAGCGTTAAGCAAGGTTTGCAAATTCGTGTCAGATCC
GGTGGTCATGACTACGAAGGTTTATCCTACGTCTCCAACGATGACGTT
CCATTCTTGATTATCGACTTGACCAACTTGAGATCCATCACTATCGATA
TCAAGGATGAAACTGCTTGGGTTCAATCTGGTGCTACCTTGGGTGAAT
TATATTACGCTATTGCTAAGAAGTCTAACGTTCACGGTTTCCCAGCTG
GTTCTTGTCCAACTGTCGGTGTTGGTGGTCACTTCTCTGGTGGTGGTT
TCGGTACCATTTTCAGAAAGTACGGTTTAGCCGCTGACAACGTCATTG
ACGCTCAAATGGTTGATGTTAACGGTAAGATCTTGAAGAACAGAACT
TTGATGGGTGAAGATTTGTTTTGGGCTATCAGAGGTGGTGGTGGTTCT
TCCTTCGGTGTTGTTACCGCTTGGAAGGTCAAGTTGGTCCATGTTCCT
CCAAAAGTTACTGTTTTCAACATTCCAAAGACCTTGGACCAAAATGC
TTCTACTTTATTCCACAAGTGGCAAATTGTTGCTGATAAGTTGCCAGG
TGAATTGTTTTTGCACTCTGTCATGGGTGTCTCTAACGCTAACTCTATT
TCTTCTTCCTCAGGTGGCGGTAGAACCGTCCTAGTCTCTTTCACCGGT
CTATACTTGGGTACCGTTGAAAACTTGTTGCCATTGATGCAAAACAAC
TTTGCTGAATTAGGTTTGCAACACAACAACTGTACTGAAATGTCTTGG
ATTCAATCTGTACTTTTCTTTGCCGGCTACGACCCAATCAACGACAGT
TTGGAGGTCTTACTTCAAAGAAACCAAACTTTCGGTTCTTTCAAGGC
CAAATCTGACTACGTTATGGAACCTATCCCAACCTCTGGTTTGGAAGG
TTTATGGAACATGTTGTTGGAAGAAAATTCTTCTCCAACTTTGATTTT
GACTCCATACGGTGGTAGAATGTCCGAAATCTCTGAATCTGAAACCCC
ATTCCCTCACAGAAACGGATCCATCTACGGTATCCAATACTTGGTTTA
CTGGGATTCTTCTGAAGAAACACCAAAGCACATCGCCTGGATGAGAA
GAGTCTACTCTTACATGGTCTTGTACGTTTCCAAGTTCCCAAGAGCTG
CTTACTTGAACTACAGAGATTTGGATATTGGTGTCAACCGTGGTAACA
CTTCTTATGACTATGAAGAAGCTAAGTCCTGGGGTCTCAAATACTTCA
AGTGTAACTTCGAAAGATTGGCCAAGGTTAAGGCTGAAGTTGACCCA
TCCAACTTCTTCAGACACGAACAATCCATTCCATTGTTGTTTTGA
>SEQ ID NO: 39 (GgDMT)
ATGGAACACTTTTATATGTCTTTATTGTTACTATTCGTTACATTGGTTTC
TTTGAGCTTATTCTTCTTGATCTTTTACCACAACAAGCACAACATGAA
CAACAACAACAATTTGCCTCCAGGTAAGATGGGTTACCCAGTTATCG
GTGAATCTTTGGAATTTTTGTCCATGGGTTGGAAGGGTCACCCTGAAA
AGTTCATTTTCGACAGAATGGTTAGATACTCCAGTGAGTTAATTAAGA
CTTCCATCTTGGGTGTTCCAACTGTCATCTTCTGTGGTCCAGCTTGTA
ACAAATTCTTGTTCTCTAACGAAAATAAGTTGGTGACTGCTTGGTGGC
CAGACTCTGTCAACAAAATCTTCCCAACCACTTCTAATTCTAAGGAA
GAATCCAAGAAGATGAGAAAGTTGTTGCCACAATTCTTGAAGCCAGA
AGCTTTGCAACGTTACGTTGGCATTATGGACACTTTAGCTCAAAGACA
CTTCGCGTCTTTGTGGGAAGAAAAGACCCATGTCACCGTTTATCCTTT
GGCTAAGAGATACACTTTCATGTTGGCCTGTAGATTGTTCATGTCTGTT
GAAGATGAAAACCACGTTGCCAAGTTCAGAGAACCATTCCATTTGTT
GGCCTCCGGTATCATCTCTGTCCCAATTGACTTGCCATGGACTCCATTC
AACAGAGGTATCAAGGCTTCAAACTTCATCAGAAAAGAATTGTTGAA
AATTATCAGACAAAGAAAGGTCGACTTAGCTCAAGGTGTCGCTTCCC
CAACCCAAGATATCTTGTCTCACATGTTGCTCACCTGTGATGACGAAA
ACGGTGAATTCATGACTGAATTGAACATCGCTGACAAGATTTTGGGTT
TGTTGATTGGTGGTCATGACACCGCTTCCGCTGCTTGCACTTTCATTG
TCAAGTACCTAGCTGAACTTCCTCACATTTACGATAGAGTCTACCAAG
AACAAATGGAAATTGCTAACTCTAAGTCTCCAGGTGAATTGCTCAAC
TGGGATGATATCAACAAGATGCGTTACTCTTGGAACGTTGCTTCTGAA
GTTATGAGAGTTGCCCCACCATTGCAAGGTGGTTTCCGTGAAGCTATT
AACGATTTCGTCTTTAATGGTTTCTCCATACCAAAGGGTTGGAAGTTG
TACTGGTCTGCCAACTCCACCCACAAGAACCCAGAATACTTCCCAGC
TCCAGAAAAGTTTGACCCAACCAGATTCGAAGGTAACGGTCCAGCCG
CTTACACCTTCGTCCCGTTCGGTGGTGGACCAAGAATGTGTCCAGGT
AAGGAATACGCTAGATTGGAAATCCTAGTTTTCATGCACAACTTGGTC
AAACGTTTTAAGTGGGAAATGTTGATTCCAGAAGAAAAGATTGTTGT
CGATCCATTGCCTATGCCAGCCAACGACCTACCAATCAGATTATACCC
ACACAACACTTGA

The nucleotide sequences provided above serve as explanatory purposes only. Any codon-optimized nucleic acid sequence obtained based on the amino acid sequences shall fall within the scope of the present disclosure.

Finally, it should be noted that the examples above are merely used to illustrate the technical solutions of the present disclosure rather than limiting the scope thereof. Although the present disclosure has been described in detail with reference to the foregoing examples, those ordinary skilled in the art will appreciate that they may still modify the technical solutions described in the foregoing examples, or make equivalent substitutions to some or all of the technical features thereof. Such modifications or substitutions do not cause the nature of the corresponding technical solutions to depart from the scope of the technical solutions of the examples of the present disclosure.

In the description of the present disclosure, terms “an embodiment”, “some embodiments,” “an example,” “a specific example,” or “some examples” or the like mean that a particular feature, structure, material, or characteristic described in the embodiment or example is included in at least one embodiment or example of the present disclosure. The appearances of the phrases throughout this specification are not necessarily referring to the same embodiment or example of the present disclosure. Furthermore, the particular feature, structure, material, or characteristic may be combined in any suitable manner in one or more embodiments or examples. In addition, various embodiments or examples described in the specification and features in various embodiments or examples may be combined by those skilled in the art without contradiction to each other.

Although explanatory embodiments have been shown and described, it would be appreciated by those skilled in the art that the above embodiments cannot be construed to limit the present disclosure, and changes, alternatives, and modifications can be made in the embodiments in the scope of the present disclosure.

What is claimed is:

1. A method for biosynthesizing glabridin, comprising:
providing a biological host;
engineering the biological host to heterologously express enzymes of a 4′-O-methyltransferase, an isoflavone 2′-hydroxylase, an isoflavone reductase, a vestitone reductase, a prenyltransferase, a pterocarpan reductase, an oxidative cyclase, and a demethylase to obtain an engineered biological host; and
culturing the engineered biological host to obtain the glabridin,
wherein the biological host is yeast,
wherein the glabridin is biosynthesized using daidzein as a substrate.

2. The method for biosynthesizing glabridin according to claim 1, wherein the engineered biological host heterologously expresses the 4′-O-methyltransferase with an amino acid sequence of SEQ ID NO: 1, the isoflavone 2′-hydroxylase with an amino acid sequence of SEQ ID NO: 3, the isoflavone reductase with an amino acid sequence of SEQ ID NO: 5, the vestitone reductase with an amino acid sequence of SEQ ID NO: 7, the prenyltransferase with an amino acid sequence of SEQ ID NO: 16, the pterocarpan reductase with an amino acid sequence of SEQ ID NO: 11, the oxidative cyclase with an amino acid sequence of SEQ ID NO: 13, and the demethylase with an amino acid sequence of SEQ ID NO: 14.

3. The method for biosynthesizing glabridin according to claim 1, wherein the engineered biological host heterologously expresses the 4′-O-methyltransferase with an amino acid sequence of SEQ ID NO: 2, the isoflavone 2′-hydroxylase an the amino acid sequence of SEQ ID NO: 4, the isoflavone reductase with an amino acid sequence of SEQ ID NO: 6, the vestitone reductase with an amino acid sequence of SEQ ID NO: 8, the prenyltransferase with an amino acid sequence of SEQ ID NO: 10, the pterocarpan reductase with an amino acid sequence of SEQ ID NO: 12, the oxidative cyclase with an amino acid sequence of SEQ ID NO: 13, and the demethylase with an amino acid sequence of SEQ ID NO: 15.

4. The method for biosynthesizing glabridin according to claim 1, wherein the yeast comprises Saccharomyces cerevisiae, Yarrowia lipolytica, and Pichia pastoris.


SEQUENCE LISTING (39 sequences)

SEQ ID NO: 1 - protein, 353 aa, Pueraria montana
  source 1..353: note = The sequence is derived from Pueraria montana var. Lobata.
MGSNNGRKASEIFQGQALLYRHMFAFVDSMCLKSIVELGIPNIIHKHGQPITLSELMSILQVPPAKVGHVQSLMRYLAHHGFFERLRIHEKDAYALTAASELLVKGTEPYLAPMVECMLDPTLSASFHQMKKWVYEEDLSVFDISLGSNLWDFLTKNPAYNEIFNEAMASDSQMSNLALRDCKLVFEGLESIVDVGGGTGTTAKIICEAFPNLKCIVFDRPQVVENLLENNNLTYVGGDMFKSIPKANAVLLKWILHDWTDKDCRKILENCKEAISNNSKRGKIIIIDIVINEKQDEHKVTELKLLFDVAMACVLNGKERNEEEWKKLFMEVGLQDYKISPLTGYLSLIEIYP

SEQ ID NO: 2 - protein, 366 aa, Glycyrrhiza glabra
MDFSTNGSEESELYHAQIHLYKHVYNFVSSMALKSAMELGIADVIHNHGKPITLPQLASALKLHPSKVGVLYRFLRLLTHNGFFAKTTVPSQNGKEGEEEETAYALTPPSKLLVKGKPTCLASIVRGALHPSSLDMWRSSEKWFNEDKELTLFESATGESFWDFLNKDSESGTLSMFQEAMAADSQMFKLALKECRHVFEGLESLVDVGGGTGGVTKLIHEEFPHLKCTVFDQPQVVGNLSGNENLKFVGGDMFKSIPPADAVLLKWVLHDWNDELSLKILKNSKEAISGKGKEGKVIIIDISIDEASGDRELTELQLDYDLVMLTMFNGKEREKKEWEKLISDAGFSSYKITPICGFKSLIEVFP

SEQ ID NO: 3 - protein, 499 aa, Glycyrrhiza echinata
MEILSLLSYSVFYLALFFIFNIVIRARKFKNLPPGPPSLPIIGNLHHLKRPLHRTFKGLSEKYGHVFSLWFGSRLVVVVSSASEFQQCFTKNDVVLANRPRFLSGKYIFYNYTTLGSTSYGEHWRNLRRITALDVLSNHRINSFSGIRRDETQRLITRLADDSSTNFAEMELSSRLYDMTFNNIMRMISGKRYYGEDCDTSDLQEASQFRDMVSELLQLSGANNKTDFMPLLRFLDFENLEKRLKDISGKTDAFLRGLIEEHRTKKERANTMIDHLLNLQDSQPEYYTDQIIKGLALAMLLAGTDSSAVTLEWSMSNLLNHPEVLKKVKDELDTHVGQDRLVDESDLPKLTYLKNVINETLRLYTPAPLLLPHSTSDECNIGGYKVPQDTIVLINAWAIHRDPELWTEATTFKPERFEKKGELEKLIAFGMGRRACPGEGLAIRAISMTLALLIQCFDWKLINGDKIDLAERDGFTLTKLVPLKAMCKSRPVINKVFKQ

SEQ ID NO: 4 - protein, 506 aa, Glycyrrhiza glabra
MEILSLLSYSVFYVALFFICNLLFQARKFKNLPPGPPSLPIIGNLHHLKRPLHRTFKGLSEKYGHVISLWFGSRLVVVVSSLSVFQECFTKNDIVLADRPRFLSGKYIFYNYTTVGSSSYGEHWRNLRRITSLDVLSTHRINSFSGVRRDETQRLIQKLAEESSTDFAEIELTSKFYDMTFNNIMRMISGKRYYGDDCDMMDMEEAKQFRAMVSELLQLSGANNKTDFMPVLRLVDFENLEKRLKKISSKTDTFLRGLIQEHRNKKQHTNTMVDHLLSLQESQPEYYTDQIIKGLALGMLLAGTDSSAVTLEWALSCLLNHPEALKKARDELETHVGQDRLLEESDLTKLPYLKNIIYETLRLYTPAPLLLPHSSSDECIIGGFKVPRETIILINAWSIHRDPQIWSEATSFKPERFEKEGELDKLIAFGLGRRACPGEALALRGISLTLGLLIQCFEWKLVGDKEIDMREESGFTLSRLIPLKAMCKARPVANKLVNTQAVSLGD

SEQ ID NO: 5 - protein, 318 aa, Medicago sativa
MATENKILILGPTGAIGRHIVWASIKAGNPTYALVRKTPGNVNKPKLITAANPETKEELIDNYQSLGVILLEGDINDHETLVKAIKQVDIVICAAGRLLIEDQVKIIKAIKEAGNVKKFFPSEFGLDVDRHDAVEPVRQVFEEKASIRRVIEAEGVPYTYLCCHAFTGYFLRNLAQLDATDPPRDKVVILGDGNVKGAYVTEADVGTFTIRAANDPNTLNKAVHIRLPKNYLTQNEVIALWEKKIGKTLEKTYVSEEQVLKDIQESSFPHNYLLALYHSQQIKGDAVYEIDPAKDIEASEAYPDVTYTTADEYLNQFV

SEQ ID NO: 6 - protein, 376 aa, Glycyrrhiza glabra
MNMIEVGGYPGNPINTKQSNAQQHKPTASLTTYQNKKKKNTHPSFFNSPYFYFNFNIPMAAENKILILGPTGAIGRHIVWASVKAGNPTFALVRKTNGPVNKPKLITAANPESKEQLLESYQNAGVTLLEGDINDHESLVKAIKQVDVVICATGRLLIDDQVKIIAAIKEAGNVKRFFPSEFGLDVDRHDSVEPVREVFEDKARIRRVIEAEGVPYTYLCCHAFTGYFLRNLAQLDATVPPRDKVVILGDGNVKGAYVTEADVGTYTIRAANDPRTLNKAVHIRLPANYLTANEVVSLWEKKIGKTLEKTYVPEEKVLKDIQESSFPHNYLLALYHSQQIKGDAVYEIDPAKDVEAYDLYSDVKYTTADEYLNQFV

SEQ ID NO: 7 - protein, 326 aa, Medicago sativa
MAEGKGRVCVTGGTGFLGSWIIKSLLENGYSVNTTIRADPERKRDVSFLTNLPGASEKLHFFNADLSNPDSFAAAIEGCVGIFHTASPIDFAVSEPEEIVTKRTVDGALGILKACVNSKTVKRFIYTSSGSAVSFNGKDKDVLDESDWSDVDLLRSVKPFGWNYAVSKTLAEKAVLEFGEQNGIDVVTLILPFIVGRFVCPKLPDSIEKALVLVLGKKEQIGVTRFHMVHVDDVARAHIYLLENSVPGGRYNCSPFIVPIEEMSQLLSAKYPEYQILTVDELKEIKGARLPDLNTKKLVDAGFDFKYTIEDMFDDAIQCCKEKGYL

SEQ ID NO: 8 - protein, 326 aa, Glycyrrhiza glabra
MAEGKGRVCVTGGTGFLGSWLIKRLLEDGYGVNTTIRSDPERKRDISFLTNLPGASERLKIFNADLSDPESFGPAIEGCVGVFHTASPIDFAVSEPEEIVTKRTVDGTLGILKACKNSKTVKRVVYTSSGAAVSWGGTEKDVLDESDWSDVDMLRSVKPYSWSYAVSKTMTEKAVLEFGEQHGLDVVTLILPFIVGRFICPKIPDSVEKALVLVLGKREQIGVTRFHMVHVEDVARAYIFLLEHPNPKGRYNCSPFIVPIEEVSELLSAKYPEYQIPTVEELKEIKGAKLPDLKSKKLVDAGFEFKYTIEDMFDDAIQCCKEKGYL

SEQ ID NO: 9 - protein, 410 aa, Cullen corylifolium
MDWGLAISSSSKAYSVTTGANLWRSKHVTNNIHYPSSCITKASQHKKKTQIEYNVLRFQRPSLGHGYNFSGGGGSTYQECNRKYAVKAVYDQPKDFELEASNPKNILDSAKKFLAAFYYFSYPYTMIGITLCAFCSSALAVEKLSDISLPFFIGVLQAVIPQLFIEIYLSGVNQLYDLEIDKINKPHLPMASGQFSFKTGVIMSAAFLALSFGFTWLTGSWPLIWNLVVIASSWTAYSIDVPFMRWKRYPLVAAMCMIATWGLALPISFFHHMQTFVLQRPIGFPRSLGFLVAFMTFYSMGIALSKDIPDVEGDKEHGINSFSVLLGQKRIFWICVSLFEMAFGVGLLGGVTSPHFWTKIITGLGNVILGSILWYQAKSFDLTDKASTGSFYMYIWKLLYAALLLMAFVR

SEQ ID NO: 10 - protein, 390 aa, Glycyrrhiza glabra
MSSLFGSNVFESISTYRSYAPKASQHKRKIQKEYNFLRSRQPSLKHIYKGIEGGSTQQECNRKYVVKTVPKPSFESDPRSIDQKDILECIKNFLDALYMFTTPYSFFSSALYIISVSFIVVEKLSDISPLFFTGVLQAVVPNLFVHIYMTGLNQLCDVEIDKINKPYLPLVSGKISFATGVIVVASCLFLSLWLGWIVGSWPSTCVVISIAMIWTAYSINVPLLRWKKYPVLAAMCIFLSLAVVNPIGYFLHMQTFVFKRPASFSRPLIFVIAFKSFFSLAVALFKDVPDIEGDQTFGVQSFVARFGKKRVFWICISLIEMAYGFALLMGATSAFLWSKIVTVLGHAVLASFVLYRAKSIDLRSKTSITSFYMLIWKLMYMEYFLMPLVR

SEQ ID NO: 11 - protein, 312 aa, Glycyrrhiza glabra
MAETTTSKSKILFIGGTGYIGKFIVEASVNAGHPTFVLIRDSTLSNPAKSPIIDKFKSLAVNLVFGDLYDHQSLVKAIKQVDVVISTVGHLQLGDQDKIISAIKESGNVKRFFPSEFGNDVDRTHAVEPAKSAFATKAKIRRTIEAEGIPYTYVSSNFFAGYFLPTLSQPGATAAPRDKVIILGDGNPKAVANKEEDIATYTIKAVDDPRTLNKILYIRPPANTLSFNELVSLWEKKIGKTLGRVYVPEEQLLKQIQESSPPINVILSIGHSVYVKGDHTNFEIEPAFGVEASALYPDVKYTTVDEYLNQFV

SEQ ID NO: 12 - protein, 329 aa, Glycyrrhiza glabra
MDSTAASKILVIGGTGYMGRFLVEASAKAGHPTFALVRHSTVTNPDKSSIIHSFNTLGVNLLLGDINDHQSLVKAIKQADVVISTVNHQYISDQYKIISAIKEAGNIKRFFPSEFGNDVVRTHGVDGAKALFDIKAKFRRTIEAEGIPHTYVVANFLTQHFLPTRSRLLAIAAPLDKVVILGDGNTKATFNTEEGVATFTIRAVDDPRTLNKILYIRPPANTLSYNDLVSLWEKKTGNTLERVYVPEEQVLRLIQESSYPLNMALSICHAAYVKGDHTNYEIEPSFGVEALELYPDVKFTTVDDYLEQNRDCTPFYLNQLIPITNGTKF

SEQ ID NO: 13 - protein, 542 aa, Glycyrrhiza glabra
MAFTYSTKKLALLSIITIFISSFPEPSTSVDLESGFLQCFSSGLGNSNSTAELVLTKNSSSYASLLQSSIRNLRFMGTSVPKPTLIVTPRDLFHIQTSIKCSVKQGLQIRVRSGGHDYEGLSYVSNDDVPFLIIDLTNLRSITIDIKDETAWVQSGATLGELYYAIAKKSNVHGFPAGSCPTVGVGGHFSGGGFGTIFRKYGLAADNVIDAQMVDVNGKILKNRTLMGEDLFWAIRGGGGSSFGVVTAWKVKLVHVPPKVTVFNIPKTLDQNASTLFHKWQIVADKLPGELFLHSVMGVSNANSISSSSGGGRTVLVSFTGLYLGTVENLLPLMQNNFAELGLQHNNCTEMSWIQSVLFFAGYDPINDSLEVLLQRNQTFGSFKAKSDYVMEPIPTSGLEGLWNMLLEENSSPTLILTPYGGRMSEISESETPFPHRNGSIYGIQYLVYWDSSEETPKHIAWMRRVYSYMVLYVSKFPRAAYLNYRDLDIGVNRGNTSYDYEEAKSWGLKYFKCNFERLAKVKAEVDPSNFFRHEQSIPLLF

SEQ ID NO: 14 - protein, 515 aa, Fusarium vanettenii
MLVDTGLGLISELQAKLGWAVLLQIVPITIVAYNLLWFIYASFFSSLRKIPGPFLARISRVWEMKKTATGNIHEIMMDLHRRHGAIVRIGPRRYDFDTMEALKIIYRIGNALPKADYYKPFGLPSFPNLFDEQNPARHSAIKKQVASLYTMTALLSYEEGVDGQTAILKEQLQRFCDQKQVIDLPRFLQYYAFDVIGVITVGKSMGMMESNSDTNGACSALDGMWHYASMMAYIPNMHAWWLRLSSLLPIEVPIKGLTEYVERRIIQYRLKAAEFGDDAALKGENNFLAKLLLMEKKGTVTPVETQQAVGLNIGAGSDTTANALSTILYYLYTNPRTLHTLREELERYVKDGPISFQQSQSMPYLQAVIKEALRLHPGVGTQLTRVVPKGGLVIEGQFFPEGTEVGVNGWALYHNKAIFGNDASIFRPERWLEANENINIGGSFAFGAGSRSCIGKNISILEMSKAIPQIVRNFDIEINHGDMTWKNECWWFVKPEYKAMIKPRRCCLSRDESLV

SEQ ID NO: 15 - protein, 481 aa, Glycyrrhiza glabra
MEHFYMSLLLLFVTLVSLSLFFLIFYHNKHNMNNNNNLPPGKMGYPVIGESLEFLSMGWKGHPEKFIFDRMVRYSSELIKTSILGVPTVIFCGPACNKFLFSNENKLVTAWWPDSVNKIFPTTSNSKEESKKMRKLLPQFLKPEALQRYVGIMDTLAQRHFASLWEEKTHVTVYPLAKRYTFMLACRLFMSVEDENHVAKFREPFHLLASGIISVPIDLPWTPFNRGIKASNFIRKELLKIIRQRKVDLAQGVASPTQDILSHMLLTCDDENGEFMTELNIADKILGLLIGGHDTASAACTFIVKYLAELPHIYDRVYQEQMEIANSKSPGELLNWDDINKMRYSWNVASEVMRVAPPLQGGFREAINDFVFNGFSIPKGWKLYWSANSTHKNPEYFPAPEKFDPTRFEGNGPAAYTFVPFGGGPRMCPGKEYARLEILVFMHNLVKRFKWEMLIPEEKIVVDPLPMPANDLPIRLYPHNT

SEQ ID NO: 16 - protein, 369 aa, Cullen corylifolium
ASQHKKKTQIEYNVLRFQRPSLGHGYNFSGGGGSTYQECNRKYAVKAVYDQPKDFELEASNPKNILDSAKKFLAAFYYFSYPYTMIGITLCAFCSSALAVEKLSDISLPFFIGVLQAVIPQLFIEIYLSGVNQLYDLEIDKINKPHLPMASGQFSFKTGVIMSAAFLALSFGFTWLTGSWPLIWNLVVIASSWTAYSIDVPFMRWKRYPLVAAMCMIATWGLALPISFFHHMQTFVLQRPIGFPRSLGFLVAFMTFYSMGIALSKDIPDVEGDKEHGINSFSVLLGQKRIFWICVSLFEMAFGVGLLGGVTSPHFWTKIITGLGNVILGSILWYQAKSFDLTDKASTGSFYMYIWKLLYAALLLMAFVR

SEQ ID NO: 17 - DNA, 1101 bp, synthetic construct
  source 1..1101: mol_type = other DNA
atggacttttcgacaaatgggtctgaggaaagtgaactttatcatgctcaaattcacttgtacaagcatgtttacaattttgtgtcttcaatggctttaaagtcagccatggagttgggcatagctgatgtcatacacaatcatggtaagccaatcaccctcccccagctggcatctgctctgaaattgcacccttctaaggttggagtactttaccgattcctccggttgctaactcacaacggcttctttgctaaaaccactgttccttctcagaatggaaaagaaggcgaggaggaggaaacggcttatgcattgactccaccatcaaaattattggtaaaaggaaagcctacttgtttggcgagcattgttagaggtgcactgcatcctagtagccttgacatgtggagaagttccgagaagtggttcaatgaagataaagagcttacgttattcgagtcagccacaggagaaagtttttgggatttcttgaacaaggacagcgaatcaggaactctctcaatgtttcaagaagcaatggcagcagactctcaaatgttcaaacttgccttgaaggaatgcaggcatgtgtttgagggtttggaatctcttgttgatgtgggtggaggaactggaggggttaccaagttaatccatgaagaatttccccatctgaaatgcacagtatttgatcaaccgcaagtagtggggaacctaagtggaaatgagaatctgaagtttgttggtggtgatatgtttaagagtataccacctgctgatgctgtcctactgaaatgggttcttcatgattggaatgatgagcttagcctcaaaatccttaaaaactccaaggaggctatttctggtaaaggcaaagaagggaaagtgataatcattgacatatcgattgatgaagcgtcaggggatagggaacttacagaattgcagttggattatgatctagtgatgctgacaatgttcaatggtaaggagcgtgagaaaaaggaatgggaaaagctcattagtgacgcaggattttcctcatataaaattacaccaatttgtggtttcaaatctttaattgaggtctttccttga

SEQ ID NO: 18 - DNA, 1521 bp, synthetic construct
  source 1..1521: mol_type = other DNA
atggaaatattgtcactattaagttatagcgtgttctacgtggccctgtttttcatctgcaacctgttgttccaagctcgcaaattcaaaaacctgccaccgggtccgccaagcctgcctatcatcggtaatcttcaccatctgaagcgtccgctgcaccgcaccttcaagggtttgtccgaaaagtacggccacgtgattagcttatggtttggcagccggctggtcgttgttgtgagcagcctgtctgtttttcaggagtgcttcaccaagaacgatattgttttggcggatcgtccgcgtttcctgtctggcaaatacattttttacaattatacgaccgtaggttctagcagctatggcgaacattggcgtaacttgcgccgtatcaccagcctggatgttctttccacccaccgcattaatagcttcagcggtgttcgtcgtgacgaaacgcagcgtttgatccagaaactggcggaggagtcctcgaccgacttcgccgaaattgaattgacttcgaagttttatgatatgaccttcaacaacatcatgcgcatgatctccggcaaacgttattacggcgacgactgtgatatgatggatatggaagaggcgaaacagtttcgtgctatggtgtcagaactcttgcaactgagcggtgcaaataacaagaccgacttcatgccggtcctgcgtctggtggatttcgaaaacctggaaaagcgcctgaagaagatcagcagcaaaaccgacacctttctgagaggcctgatccaagagcaccgcaacaagaagcaacacaccaataccatggttgaccacctgctgtccctgcaagagtctcagccggagtactacactgaccagattatcaaaggtttagcgttaggtatgctgttggcgggcaccgacagctccgcagtcaccttggagtgggcactgagctgcctgctgaatcatccggaggcgcttaagaaggcgcgtgacgagctggaaacgcacgtgggccaggatcgtctcttagaggagtccgacctgacgaaactgccatatctgaagaacattatttacgaaacgctgcgtttatatactccggcaccgctgttgctgccgcattcttcatccgatgaatgtattattggtggttttaaggtgccgcgtgaaaccatcatcctgatcaacgcgtggagcattcatcgtgatccgcagatctggtcggaagctaccagttttaaaccggaacgtttcgagaaggagggtgaactggacaagctgatcgcatttggtctcggtagacgtgcctgcccgggcgaggctttggccctgcgcggtattagcctgacgctgggcctgttgattcaatgttttgagtggaaactggttggtgacaaagaaatcgatatgcgcgaagagagtggctttaccctgagccgtctgatcccgctgaaagctatgtgcaaagctcgcccggttgcgaacaaactggtaaatacccaggcggtgtccctcggggattaa

SEQ ID NO: 19 - DNA, 1131 bp, synthetic construct
  source 1..1131: mol_type = other DNA
atgaacatgattgaagtcggtggttacccaggtaacccaattaacaccaaacaatctaatgcccaacaacacaagccaaccgctagcttgactacttaccaaaacaagaagaaaaagaacactcatccatccttctttaactctccatacttctacttcaacttcaacatcccaatggctgctgaaaacaagatcctgatcttaggtccaactggtgccattggacgtcatatcgtttgggcttccgttaaagccggtaacccaacctttgctttggttagaaagaccaacggtccagttaacaaaccaaagttgatcactgctgccaatccagaatccaaggaacaattactagaatcttaccaaaacgctggtgtcactttgttggaaggtgacattaatgaccacgaatctttggttaaggctatcaagcaagtcgatgtcgtcatctgtgctaccggtagattattgatcgatgaccaagttaagatcattgcagccatcaaggaagccggtaacgttaagagattcttcccatctgaattcggcttggacgttgacagacacgactctgttgaacctgtccgtgaagtcttcgaagataaggctagaattagaagagttatcgaagctgaaggtgttccatacacttacttatgttgtcacgctttcaccggttactttttgagaaacttggctcaattggatgctactgttcctccaagagacaaggttgttattttgggtgatggtaacgtcaagggtgcttacgttactgaagctgacgttggtacatatactatcagagctgctaacgatccaagaaccttgaacaaggctgttcacattagattgccagctaactatttgaccgccaacgaagttgtctccttgtgggaaaagaagattggtaagaccttggaaaagacctacgtcccagaggaaaaagtcttgaaggacatccaagaatcctctttcccacacaactacttgttggccctataccactctcaacaaattaagggtgacgctgtctacgaaatagacccagctaaggatgtcgaagcttacgacttgtactcagatgtaaagtacaccactgctgatgaatacttgaaccaattcgtctga

SEQ ID NO: 20 - DNA, 981 bp, synthetic construct
  source 1..981: mol_type = other DNA
atggccgagggcaagggccgggtatgtgtcacgggcggcaccggcttcctgggatcgtggctgatcaagcgtttgctcgaggacggctacggcgtcaacaccacgatccggagtgacccggagcgcaagcgggacatcagcttcctcaccaacctgcccggggcgagcgagcggctgaagatcttcaacgccgacctgtccgaccccgaatccttcggccccgcgattgaaggctgcgtgggtgtcttccacaccgcctccccgatcgacttcgccgtctcggagcccgaggagatcgtgaccaagcgcaccgtcgacgggacgctgggcatcctcaaggcctgcaagaactccaagaccgtcaagcgggtcgtgtacacgtccagcggcgccgcggtcagctggggcgggaccgagaaggacgtgctcgacgagagcgactggtcggatgtggacatgctgcgctcggtgaagccgtactcctggtcctacgccgtgagcaagacgatgaccgaaaaggcggtgctggagttcggcgagcagcacggcctggacgtcgtcaccctgatcctgccgttcatcgtcggccgcttcatctgccccaagatccccgactccgtcgagaaggcgctggtcctggtgctcggcaagcgcgagcagatcggcgtgacccggttccacatggtccacgtcgaggacgtggccagggcctacatcttcctgctggagcacccgaacccgaaggggcgctacaactgctcccccttcatcgtgccgatcgaggaggtctcggagctcctctccgcgaagtacccagagtaccagatcccgacggtggaggaactcaaggagatcaagggtgccaagctcccggacctcaaatcgaagaagctggtcgacgcggggttcgagttcaagtacaccatcgaggacatgttcgacgacgccatccagtgctgcaaggagaagggatacctgtaa

SEQ ID NO: 21 - DNA, 1173 bp, synthetic construct
  source 1..1173: mol_type = other DNA
atgtcttctttattcgggtcaaatgtatttgagagtatatcaacatatcgtagctatgctccaaaagccagtcaacataaaaggaagattcagaaagaatataatttccttcgctcccgacagccttcattaaagcacatttacaagggaattgagggtggaagtacacaacaagagtgcaataggaaatatgttgtaaaaacagtgcccaagccaagctttgaatcagatcctaggtctatagatcaaaaggacatcttggaatgcattaagaattttcttgatgctttatatatgtttactactccttattcttttttctcctcagcgttgtacattatctccgtttctttcatcgttgtggagaagctttccgacataagcccgctgttcttcactggtgtgctccaagcagttgttccaaatctttttgttcacatatacatgacaggactaaaccagctttgtgatgttgaaattgacaagatcaacaaaccctaccttcccctggtctcaggaaaaattagttttgcaaccggggttattgttgtcgcgtcatgcctgttcctatcgctttggcttggatggattgtgggctcttggccatcaacttgtgtcgtgatatctatcgctatgatttggactgcttactctatcaatgtacctctcctcagatggaagaagtatccagtcctggcagcaatgtgtatatttttatctcttgctgtagtgaaccctattggttatttcttgcatatgcaaacatttgtgttcaagagacctgcttcattttcaaggccgctcattttcgtgattgctttcaaaagtttttttagtttggcagtggcattatttaaggatgttcctgatattgaaggtgatcagacctttggcgttcagtcgtttgttgccagatttggaaagaaaagagttttctggatatgtatttctctcatcgaaatggcatatggttttgctttgttaatgggggctacttccgcttttctatggtccaaaatagttacagtattgggccatgctgttcttgccagttttgtactatatagagcaaaaagcattgatttgcgtagcaaaacgagcattacctctttttacatgttgatatggaaactgatgtatatggagtattttttgatgccattggtccggtga

SEQ ID NO: 22 - DNA, 939 bp, synthetic construct
  source 1..939: mol_type = other DNA
atggctgaaacaactacctcaaaatcaaagattcttttcattggaggaactggctacattgggaaatttattgttgaagcttctgttaatgctggtcatccaacttttgtgttaataagagattcaactctgagcaatccagcgaagtctcccataattgataaattcaagagtctcgcagtgaacctcgtttttggggacttgtatgatcaccagagtcttgtgaaagcaatcaaacaagttgatgttgtgatctccaccgttggacatcttcagcttggagatcaagataagataatatcagccattaaagagtctggtaatgttaagaggtttttcccttctgaatttgggaatgatgtcgaccgtacacatgctgtagagcccgctaagtctgcctttgcaaccaaggcaaaaatccgcaggacaatagaggccgagggaattccatacacttacgtatcgtccaacttctttgctggatatttcttgcctactctatctcaacctggtgcaactgccgctccaagagacaaggttataatcttaggggatggaaaccctaaagctgtcgctaacaaggaggaagatattgctacatatacaatcaaggcggtggatgatccaagaacattgaacaaaatactttatattcggccgccggcaaatactttatctttcaatgaattggtgagtctgtgggaaaagaaaattggtaaaactcttggccgagtttatgtgccagaagagcagctcttgaagcagattcaagaatcgagtcctccaattaatgtcatactaagcatcggccactcagtatatgttaaaggtgatcatacaaattttgagattgagcctgcatttggtgtcgaagcatctgcactgtatcctgacgtaaagtacacgacggttgacgaatatttgaatcaatttgtttga

SEQ ID NO: 23 - DNA, 1629 bp, synthetic construct
  source 1..1629: mol_type = other DNA
atggcttttacgtacagtaccaagaaacttgcactgctctcaatcattactattttcatctccagctttcctgaaccatctacatcggttgaccttgagtcaggcttccttcaatgttttagttcaggattgggcaacagtaacagcacagcggagttggtgctaacaaaaaattcttcctcttatgctagcctgcttcagtctagtattagaaacttgcggttcatgggaacatctgtaccgaagcctacattgatagtgactcctagagatctctttcatatccaaacaagcatcaaatgctcagttaaacaaggattacagatccgtgtaaggtccgggggacatgattacgaaggactctcctacgtttctaatgatgatgtgccatttttgataattgatcttactaatctgcgaagtattaccattgacattaaggatgaaaccgcttgggtccaaagtggagccactcttggagagctatattatgcaattgctaagaagtcgaatgttcacggcttccctgctggttcatgccctacggtaggggttgggggccatttttcaggtggtggatttggaactatatttagaaagtacgggctggccgctgataatgtgattgatgctcaaatggtggatgttaatggaaagatacttaagaataggacattgatgggtgaagatttgttttgggcaatacgggggggtggtggctcatcatttggggtcgttactgcttggaaagttaagttagttcatgttcctccaaaagtaactgtcttcaatatccccaaaacactagatcaaaatgcaagcacccttttccacaaatggcaaattgttgcagataaattgccaggagaactattcttgcattccgtgatgggcgtctctaatgccaattctatttcttcttcctcgggtggaggaagaacagtgcttgtcagctttactggattatatctgggaactgttgaaaatctcttacctctaatgcagaacaattttgctgaattgggtcttcaacacaataactgtacagaaatgagttggattcagagcgtacttttctttgcaggatatgatccaatcaacgattctttagaagtactcctgcagaggaaccagacatttggttctttcaaagcaaaaagcgactatgtgatggagcccataccaacttcagggttggagggcctttggaatatgttattggaagagaatagtagtccaactttgattctcacgccatatggtggtaggatgtctgagataagtgagtctgaaactccatttcctcatcgtaatggttcaatttatggtatacagtatcttgtttattgggattcatctgaggaaactcccaagcatattgcatggatgaggagagtttattcttacatggtactttatgtatcaaagtttccgagggcggcttatttgaattacagagacttagacatcggtgttaaccgagggaatacctcctacgactatgaagaagccaaatcgtggggtctgaaatacttcaaatgtaactttgagagattggctaaggtgaaggcagaggttgatccttcaaacttcttccgccatgaacaatccattcctctcctgttttga

SEQ ID NO: 24 - DNA, 1446 bp, synthetic construct
  source 1..1446: mol_type = other DNA
atggagcatttctacatgtctctactacttttgtttgtaacactggtaagcctttctctctttttcttgattttctatcataacaagcacaatatgaataataacaacaatctaccccctgggaagatgggttatcctgttattggagaatctcttgagtttttgtcaatgggatggaaggggcatcctgagaagttcatatttgacaggatggttcgttacagtagtgagttgatcaaaacatcaatcttaggagtcccaactgtgatcttttgtggaccagcttgtaataagttccttttcagtaatgagaacaaattggtcactgcttggtggccagattctgtaaataagatatttcccacgacttcaaatagcaaagaggaatccaagaaaatgcggaaattgcttcctcaatttcttaagcctgaagctttacagaggtatgttggcataatggatactctggctcagcgacattttgctagtttgtgggaggagaaaactcatgttactgtttacccgctggcaaaacggtacacattcatgcttgcttgccgcctattcatgtctgttgaagatgagaatcatgtggcgaagtttcgcgaacctttccacttgcttgcatctggtatcatatcagtcccaattgatctaccatggacaccatttaacagaggcatcaaggcatcaaattttattaggaaggaactgttaaagataattagacagagaaaagtagatttggcccagggcgtagcgtcccctactcaagatattttaagccatatgttgttaacatgtgatgatgaaaacggtgaatttatgacagaattgaatattgctgataaaatactcggtctcctcattggtggtcatgacacagcatctgccgcatgcactttcatcgtgaagtatttggctgaacttccacacatttatgatagagtttatcaagaacaaatggaaatagctaattcaaaatctccaggagagctgcttaattgggatgacattaacaagatgcgttattcatggaacgttgctagcgaagtcatgagggtggcccctcctcttcaagggggttttcgagaagcaattaatgattttgtgttcaatggattttcgattcccaagggttggaaactctattggagtgcaaactccacgcataaaaatcccgagtattttccagccccggagaaattcgatccaaccaggtttgaaggaaatggccctgctgcatacacctttgttccatttggaggagggccaagaatgtgtcctggaaaagaatatgctagattagagatattggtctttatgcataatcttgttaagagattcaaatgggaaatgctaattccagaagaaaaaatcgttgtggacccgttacctatgccagcaaatgacctgcccattcgtctttaccctcacaacacctga

SEQ ID NO: 25 - DNA, 1062 bp, synthetic construct
  source 1..1062: mol_type = other DNA
atgggttccaacaacggtcgtaaagcttctgaaattttccaaggtcaagctttgctatacagacacatgtttgctttcgtcgactccatgtgtttgaagtccattgtcgaattaggtatcccaaacatcattcacaagcatggtcaaccaatcaccttatccgaattgatgtccatcttgcaagtccctccagctaaggttggtcacgttcaatctttgatgagatatctggctcaccacggtttcttcgaaagattgagaattcacgaaaaggatgcttacgctctgactgctgcttccgaattgttagtcaagggtaccgaaccatacttggctccaatggttgaatgtatgttggatccaactttgtctgcttctttccaccaaatgaagaagtgggtttacgaagaagatttgtcagttttcgacatctccttgggttctaatttatgggatttcttgaccaagaacccagcctacaacgaaatcttcaacgaagctatggcttctgattctcaaatgtctaacttggccttgagagattgcaagttagtctttgaaggtttggaatccatcgtggatgtcggtggtggtactggtactacagccaaaattatttgtgaagccttcccaaacttgaagtgtatcgtctttgacagaccacaagttgttgaaaacttgttagagaacaacaacttgacctacgttggtggtgacatgttcaagtctattccaaaggccaacgctgtcttattgaaatggattctacatgactggactgacaaggactgtcgtaagattttggaaaactgtaaggaagctatctctaacaatagtaagagaggtaagatcatcatcattgacattgttatcaacgaaaagcaagacgagcacaaggtcactgaattgaaattgttgttcgatgttgctatggcctgtgttttgaacggtaaggaaagaaacgaagaagaatggaagaagcttttcatggaagttggattgcaagactacaaaatttctccattgaccggttacttgtctttgatagaaatctacccatga

SEQ ID NO: 26 - DNA, 1500 bp, synthetic construct
  source 1..1500: mol_type = other DNA
atggaaattttgtctttgttgtcctactcagttttctatttggctttgttcttcatcttcaacatcgttatcagagctagaaagttcaagaatttgcctccaggtccaccatctttgccaattatcggtaacctccaccacttaaagagaccattgcatagaaccttcaagggtttgagtgaaaagtacggtcacgttttctccttgtggttcggttccagactggtcgtcgttgtctcctccgcctctgaattccaacaatgtttcaccaagaacgatgttgttctagccaacagaccaagattcctaagcggtaaatacattttctacaactacaccactttgggttctacctcttacggcgaacattggagaaacttacgtagaatcactgctttggatgtcttgtccaaccatagaattaactctttctctggtatcagaagagacgaaactcaaagattgatcaccagattagctgatgactcttccaccaacttcgctgaaatggaattgtcttctagattgtacgacatgactttcaacaatatcatgagaatgatctccggtaagagatactatggcgaagactgtgatacttctgacttgcaagaagcttctcaattcagagacatggtttccgaattgttgcaattgtctggtgctaacaacaaaactgatttcatgccattgttgagattcttggacttcgaaaacttggaaaaaagattgaaggacatttctggtaagaccgatgcttttttgagaggattgattgaagaacacagaactaagaaggaaagagccaacaccatgattgaccacttgttaaatttacaagattcccaaccagaatactacaccgaccaaatcatcaagggtttagccttggccatgttgttggctggtactgactcctctgctgtcactttggaatggtccatgtctaacttgttgaaccacccagaagttttgaagaaagttaaggatgaattggacactcacgtcggtcaagatcgtttagtcgacgaatctgatttgccaaagttgacttacttgaagaacgttatcaacgaaaccttaagattatacactccagctccattgttgctgccacactctacttccgatgaatgtaacattggtggttacaaggttcctcaagacaccattgttttgatcaacgcttgggctattcaccgtgatccagaattatggactgaagctaccacatttaagccagaacgttttgaaaagaaaggtgaattggaaaagttgattgctttcggtatgggtagaagagcttgtccaggtgaaggtctagccattcgtgcaatctctatgaccttggctttgttgattcaatgctttgactggaagttaatcaacggtgacaagatcgacttggctgagagagatggtttcactttgaccaaattggtcccattgaaggctatgtgtaagtctcgtccagttattaacaaggtctttaagcaatga

SEQ ID NO: 27 - DNA, 957 bp, synthetic construct
  source 1..957: mol_type = other DNA
atggctaccgaaaacaagatcttgattttgggtccaactggtgccatcggtagacacattgtctgggcttccatcaaggctggtaaccctacctacgccttagtcagaaagaccccaggtaatgttaacaagccaaagttgatcactgctgctaacccagaaaccaaagaagaattgattgacaactaccaatctttgggtgtcatcttgttggaaggtgacatcaacgaccacgaaacattggtcaaggctattaagcaagtcgatattgttatctgtgctgccggccgtttattgatcgaagaccaagtcaagataatcaaagctatcaaggaagcaggtaacgtcaagaagttcttcccatctgaattcggtttagacgttgacagacatgatgctgttgaaccagttagacaagtttttgaagaaaaagcttccattagaagagttattgaagctgaaggtgttccatacacttatttgtgttgtcacgctttcaccggttacttcttgagaaacttggctcaattggacgccaccgaccctccaagagacaaggttgttattttaggtgatggtaacgttaagggtgcttacgttactgaagctgatgttggtacttttactatccgtgctgctaatgacccaaacactttaaacaaagccgtccatatcagattgccaaagaactacttgactcaaaacgaagtcattgctttgtgggaaaagaagattggtaagaccttggaaaagacttacgtctcagaagaacaagttttgaaggatatccaagaatcttctttcccacacaactatctactggctttgtaccactctcaacaaatcaagggtgatgctgtatacgaaattgatccagctaaggacattgaagcctccgaggcttacccagacgtcacctacaccactgccgatgaatacttgaaccaattcgtctga

SEQ ID NO: 28 - DNA, 981 bp, synthetic construct
  source 1..981: mol_type = other DNA
atggccgaaggtaaaggtagagtctgtgtcaccggtggtactggcttcttgggttcctggattataaagtctttattagaaaacggttactccgtcaacaccactatcagagcagatccagagagaaagagagatgtcagtttcttgactaatttgccaggtgcctctgaaaaattgcacttcttcaacgccgacttgtctaacccagattcttttgctgctgctattgaaggttgtgttggtatcttccacactgctagcccaatcgactttgctgtttccgaaccagaagaaatcgttaccaagagaaccgttgacggtgctcttggaatcttgaaagcttgtgttaactctaagactgtcaagcgtttcatttacacctcttctggttctgctgtttctttcaatggtaaggacaaggatgttttggacgaatccgactggtctgacgtcgacttgttgagatctgtcaagccattcggttggaactacgctgtctccaagaccctagctgaaaaggctgtattggaattcggtgaacaaaacggtattgacgttgttactttgattttgcctttcattgtcggtcgtttcgtctgccctaagttgccagactccattgaaaaggctttggtcctagttttgggtaagaaagaacaaattggtgttaccagatttcacatggttcacgttgatgacgtcgccagagcccatatctatttattggaaaactccgttccaggtggtagatacaactgttctccattcatcgttccaattgaagaaatgtcacaattgttgtccgctaagtacccagaataccaaatcttgactgtggatgaattgaaggaaatcaagggtgctagattaccagatttgaacactaagaagttagtcgatgctggtttcgacttcaagtacacaatcgaagacatgttcgatgatgctatccaatgttgtaaggaaaagggttacttgtga

SEQ ID NO: 29 - DNA, 1110 bp, synthetic construct
  source 1..1110: mol_type = other DNA
gcatctcaacacaagaagaagactcaaatcgaatacaacgtcttgagattccaaagaccatccttgggtcacggatacaacttctccggtggtggtggctccacttaccaagaatgtaacagaaagtacgccgttaaggctgtctacgaccaaccaaaggacttcgaattggaagcttccaacccaaagaacattttggactctgccaagaaattcttggctgctttttactacttcagttacccatacaccatgattggtatcactttgtgcgctttctgttcctctgctttagctgtcgaaaagttgtctgatatctccctccctttcttcatcggtgtcttgcaagctgttatcccacaattgtttattgaaatttacttgtctggtgttaaccaattatatgacttggaaatcgacaagattaacaagccacatttgccaatggcttctggtcaattctctttcaagaccggtgtcatcatgtctgccgcctttttggctttgtccttcggtttcacctggttgaccggatcttggccattgatctggaacttggttgttattgcctcctcctggactgcttactctattgacgttccattcatgagatggaagagatacccattagttgctgctatgtgtatgatcgctacctggggtctggctttaccaatctcttttttccaccacatgcaaactttcgttttgcaacgtcctattggtttcccaagatctttgggtttcttggttgctttcatgactttttattccatgggtattgctttgagcaaggatattccagatgttgaaggtgacaaggaacatggtatcaattctttctcagtcttgttgggtcaaaagcgtatcttctggatctgtgtctcattgtttgaaatggctttcggtgttggtctattaggtggtgttacttctccacacttctggaccaagatcattaccggtctaggtaacgtcattttgggttccatcttgtggtaccaagctaagtctttcgatttgacagataaagcttctactggttctttctacatgtacatctggaaattgttgtacgctgccttattgttgatggccttcgtcagatga

SEQ ID NO: 30 - DNA, 939 bp, synthetic construct
  source 1..939: mol_type = other DNA
atggccgaaaccaccacttctaagtctaaaatattgtttattggtggtaccggttacattggcaagttcattgttgaagccagtgtcaatgccggtcacccaactttcgtcttgattagagatagcactttgtctaacccagcgaagtccccaatcattgataaattcaagtctttggctgtcaacttggttttcggtgacttgtacgaccaccaatctttggtcaaggccatcaaacaagttgatgtagtgatctccacagtcggtcatttgcaattgggtgaccaagacaagattatttccgctatcaaggaatccggtaatgtcaagagattcttcccatcagaattcggtaacgacgttgacagaacccacgctgtcgaaccagctaaatccgctttcgctaccaaggctaagatcagacgtactatcgaagctgaaggtatcccatacacttacgtttcttccaacttttttgctggttacttcttgccaaccttatcccaaccaggtgctactgctgctccaagagacaaggttatcatcttgggtgacggtaacccaaaggctgttgctaacaaggaagaagatattgctacttacactatcaaggcagtcgatgacccacgtaccttaaacaagattttgtacatcagaccaccagccaacactctatctttcaacgaattggtttccttatgggaaaagaagatcggtaagaccttgggtagagtctatgtcccagaagaacaactgttgaagcaaattcaagagtcttctccacctatcaacgttatcttatctattggtcactctgtttacgtcaagggtgaccacaccaacttcgaaattgaacctgctttcggtgttgaagcctctgctttgtatccagatgttaagtacaccactgttgatgaatacttgaaccaattcgtttga

SEQ ID NO: 31 - DNA, 1629 bp, synthetic construct
  source 1..1629: mol_type = other DNA
atggctttcacttactccactaagaagttggctttgttgtctatcatcactatattcatctcctccttcccagaaccatctacttctgttgatttggaatctggtttcttgcaatgtttctcttctggtctaggtaactctaactcaactgctgaattggtcttgaccaagaactcttcctcttacgcctccctgttgcaatcttccattcgtaatctgagattcatgggtacctcggtcccaaagccaaccttgattgtcactccacgggatttgttccatattcaaacctctattaagtgttctgttaagcaaggtttgcaaatcagagttagatcaggtggtcacgactacgaaggtttgtcctacgtctccaacgatgacgttccattcctgattatcgacttgaccaacttgcgtagtatcaccattgacatcaaggacgaaactgcttgggttcaatctggtgcaactttgggtgaattgtactacgctattgccaagaagtctaacgtccacggtttcccagctggttcttgtccaaccgtcggtgttggtggtcacttctctggtggtggtttcggtaccatcttcagaaagtacggtctagccgctgataacgttatcgacgctcaaatggttgatgtcaacggtaagatcttgaagaacagaaccttgatgggtgaagacttgttctgggctatcagaggcggtggtggttcttcctttggtgtcgtcaccgcttggaaggttaaattggtccatgttccaccaaaagtgacagttttcaacatccctaagaccttggaccaaaacgcttctaccttgttccacaagtggcaaatcgttgctgacaagttaccaggtgaattgtttttgcacagtgtcatgggtgtttccaacgccaactccatttcctcatcttccggtggcggtcgtactgtcttagtttctttcaccggcttgtacttgggtactgttgagaacctattgccattgatgcaaaacaactttgctgaattaggtttacaacacaacaactgtactgaaatgtcctggattcaatccgttttattctttgccggttacgacccaatcaacgactctttggaagttttgttacaaagaaaccaaactttcggttccttcaaggccaagtctgactacgttatggaacctattccaactagcgggttggaaggtttgtggaacatgttgctcgaagaaaatagctctccaactttgatcttgactccatacggtggtagaatgtctgaaatttctgaatctgaaaccccgttcccacacagaaatggttccatctacggtatccaatacttagtttactgggattcctccgaagaaaccccaaagcatattgcttggatgagaagagtctactcttacatggttttgtacgtttctaagttcccaagagctgcttacttgaactacagagatttggatattggtgttaacagaggtaacacttcctatgactatgaagaagccaaatcttggggtttaaagtatttcaaatgcaatttcgaaagattggctaaagtcaaggctgaagtcgatccttctaacttcttcagacacgaacaatctattccattgttattttga

SEQ ID NO: 32 - DNA, 1548 bp, synthetic construct
  source 1..1548: mol_type = other DNA
atgctcgttgacactggtttaggattgatttctgaattgcaagccaagttgggttgggctgttttgttgcaaattgtcccgatcaccatcgtcgcttacaacttactgtggttcatctacgcttccttcttttcctctttgagaaagattccaggtccattcttggctcgtatctcccgtgtctgggaaatgaagaagactgctaccggtaacattcacgaaatcatgatggacttgcacagacgtcacggtgctatcgtcagaatcggtccaaggagatatgacttcgacaccatggaagctttgaagatcatttacagaattggtaatgccttgcctaaagctgactactacaagccattcggtttgccatctttcccaaacttgtttgatgaacaaaacccagctagacattccgctatcaagaaacaagttgcttctttgtacactatgactgctttattgtcttacgaagaaggtgtcgatggtcaaaccgccatcttgaaggaacaattgcaaagattctgtgaccaaaagcaagtcattgatttgccaagattcttgcaatactacgctttcgatgttatcggtgttatcactgttggtaagtctatgggtatgatggaatccaactctgatactaatggtgcttgctctgctttggacggtatgtggcactacgcctccatgatggcttacatccctaacatgcacgcttggtggttgagattgagctctcttttgccaatcgaagttccaattaagggtttgactgaatacgttgaaagaagaatcatccaatacagattaaaggctgctgaattcggtgatgatgctgccttgaaaggtgaaaacaactttttggctaagttgttgttgatggaaaagaagggtacagtcactccagtggaaactcaacaagctgtcggcttgaacattggtgctggttctgacactactgcgaacgctttgagtaccatcttgtattatttatacaccaacccaagaaccctgcacactttgagagaagagttggaacgttacgtcaaggatggtccaatctctttccagcaatctcaatccatgccatacttgcaagctgtcatcaaggaagccctaagattacatccaggtgttggtacccaattgaccagagttgttccaaagggtggtttagtcattgaaggtcaattcttcccagaaggtaccgaagtcggtgttaacggttgggctctataccacaacaaggccatttttggtaacgatgcctccatcttcagaccagaaagatggttagaagctaacgaaaacattaacatcggtggttctttcgccttcggtgccggttccagatcttgtatcggcaagaatatcagcattttggaaatgtccaaggctattccacaaattgttagaaacttcgacattgaaattaaccacggtgacatgacctggaagaacgaatgttggtggttcgttaaaccagaatacaaggctatgattaagccaagaagatgttgtttgtctagagacgaatctctggtttga

SEQ ID NO: 33 - DNA, 1101 bp, synthetic construct
  source 1..1101: mol_type = other DNA
atggacttctctaccaacggttctgaagaatcagaattataccacgctcaaatccatttatataagcacgtttacaacttcgtttcttccatggctctgaagtctgctatggaattgggtattgccgatgttattcacaatcacggtaagccaattactttgccacaattggctagtgctttgaagttgcacccatctaaggtcggcgttttatacagattcttgagattattgactcacaatggttttttcgctaagaccactgttccgtcacaaaacggtaaggaaggtgaggaagaagaaaccgcttacgctttgaccccaccttccaaattgttggtcaagggtaagccaacttgtttggcctccattgtcagaggtgctttgcatccaagctctcttgacatgtggagatcttctgaaaagtggtttaacgaagacaaggaattaacattgttcgaatccgccactggtgaatctttctgggatttcttgaacaaggactccgaatctggtactttgtccatgttccaagaagccatggctgctgactctcaaatgttcaagttggccttgaaggaatgtagacacgtctttgaaggtttggaatctttagtcgatgttggtggaggtaccggtggtgtcaccaagttgatccacgaagaattcccacatttgaaatgcaccgtcttcgaccaaccacaagttgttggtaacttgtctggtaacgaaaacctaaaattcgtcggtggtgacatgtttaagtccatcccaccagctgatgctgtcttgttgaaatgggttttgcacgactggaacgatgaactatctttgaagattttgaagaactccaaggaagctatctccggtaagggtaaagaaggtaaggttatcatcattgatatttctatcgacgaagcttccggtgatcgtgaattgactgaattgcaattggactacgatttggttatgttgaccatgttcaacggtaaggaaagagaaaagaaggaatgggaaaaattaatctctgacgctggtttctcctcttacaagatcactccaatctgtggtttcaagtccttgattgaagttttcccatga

SEQ ID NO: 34 - DNA, 1521 bp, synthetic construct
  source 1..1521: mol_type = other DNA
atggaaatcttgtccttgttgtcttactccgttttttacgttgctttgttcttcatctgtaacttgttattccaagctagaaagttcaagaatttgccacctggtccaccatctctcccaatcattggtaacttacatcatttgaagagaccattgcacagaactttcaagggattgtctgaaaagtacggtcacgttatctctttgtggttcggttctagattggttgtcgtcgtgtcttccttatctgtcttccaagaatgcttcaccaagaacgatattgttctcgctgacagaccaagattcttgagtggtaaatatatcttctacaactacaccactgtcggttcctcttcctacggtgaacactggagaaacttaagaagaatcacttccttagatgtcttgtccacccacagaattaactctttctctggtgttagaagagatgaaacccaaagactgatccaaaagttggccgaagaatcatccactgattttgctgaaattgaattgacctccaagttttacgatatgactttcaacaacatcatgagaatgatttccggtaagagatactacggcgatgactgtgacatgatggacatggaagaagctaagcaattcagggccatggtttccgaattgctacaattgtctggtgctaacaacaagaccgacttcatgccagtcttgagattggtcgatttcgaaaacttggaaaaacgtttaaagaagatctcctctaagactgacaccttcttgcgtggtttgatccaagaacacagaaataagaagcaacacaccaacaccatggttgaccacttgctgtctttgcaagaatctcaaccagaatactatactgaccaaatcatcaagggtttagccttgggtatgttgttggctggtaccgattcctcagctgttactttggaatgggctctatcttgtttgttgaaccacccagaagccttgaagaaggcaagagacgaattagaaacccatgtcggtcaagatcgtttgttggaagaatccgatttgacaaaattgccatacttgaagaacattatctacgaaactttgagattgtacactccagctccattattgttgcctcactcttcttctgacgagtgtatcatcggtggtttcaaggttccaagagaaactattattttgattaacgcttggtctattcaccgtgacccacaaatctggtctgaagctacttctttcaagccagaaagatttgaaaaggaaggtgaattggacaaattaattgctttcggtctgggtagaagagcttgtccaggtgaagctttggccttgagaggtatctctttgaccttgggtctattgattcaatgtttcgaatggaagttggttggtgacaaggaaattgacatgcgtgaagaatctggtttcactttatccagattaattccattgaaggctatgtgtaaggctcgtccagttgccaacaaattagttaacactcaagctgtctctttgggtgattga

SEQ ID NO: 35 - DNA, 981 bp, synthetic construct
  source 1..981: mol_type = other DNA
atggctgaaggtaagggtagagtctgtgtcaccggtggtactggtttcctaggttcatggttaatcaagcgtttattggaagacggttacggcgtcaacactaccatcagatccgacccagaaagaaagagagacatttctttcttgactaacttgccaggtgcctccgaaagattaaaaatcttcaatgctgacttgtccgacccagaatccttcggaccagctattgaaggttgtgttggtgtcttccacactgcttccccaattgacttcgccgtcagtgaaccagaagaaattgtcaccaagcgtactgttgatggtactttgggtatcttgaaggcttgcaagaactccaagactgttaagagagttgtctacacctcttctggtgctgctgtctcttggggtggtaccgaaaaggatgtcttggacgaatctgactggtccgatgtggacatgttgagatctgtcaagccatactcttggtcttacgctgtttccaagaccatgactgaaaaggccgtcttggaattcggtgaacaacacggtttggatgttgttacattgattttgccattcattgtcggtagatttatctgtccaaagatccctgactctgttgagaaagctttggtcctcgttttgggtaagagagaacaaatcggtgttaccagattccacatggttcatgttgaagacgttgctagagcttacatctttttgcttgaacacccaaacccaaaggggagatacaactgttctccatttattgttccaatcgaagaagtttctgaattgttgtctgccaaatacccagaataccaaattccaaccgttgaagaattaaaagaaatcaagggtgctaagttgcctgatttgaagtctaaaaagttagttgatgctggtttcgaattcaagtacactattgaagatatgttcgacgatgccatccaatgttgtaaggaaaagggttatttgtga

SEQ ID NO: 36 - DNA, 1173 bp, synthetic construct
  source 1..1173: mol_type = other DNA
atgtcttctctatttggctctaacgttttcgaatccatttccacttacagatcttacgctccaaaggcttctcaacacaagagaaagatccaaaaggaatacaacttcttaagatccagacaaccatctttgaagcacatttacaagggtatcgaaggtggttccactcaacaagaatgtaacagaaagtacgtcgttaagaccgttccaaaaccatcctttgaatctgacccaagatccatcgaccaaaaggatattttggaatgtatcaagaactttttggatgccctttatatgttcaccactccatactcgttcttttcctccgctttgtacatcattagtgtttctttcatcgttgttgaaaagttgtctgacatttccccattgttcttcaccggtgtcttgcaagctgtggttccaaacttgttcgtccatatctacatgacaggtttgaaccaattgtgtgatgtcgaaatcgacaagatcaacaagccttacttgccattggtctctggtaaaatttcctttgccaccggtgtaattgtcgttgcttcttgtctcttcttgtccttgtggttgggttggatcgtcggttcttggccttctacctgtgtcgtcatctccatagccatgatctggactgcttactctatcaatgttccattattgagatggaagaaatacccagttttggctgctatgtgtattttcttgtctttggccgtcgttaacccaattggttacttcttacacatgcaaactttcgtcttcaagcgtccagcttccttctctagaccattgatcttcgttatagctttcaaatctttcttctctttagccgtcgctttattcaaggacgtcccagacattgaaggtgatcaaaccttcggtgtccaatcttttgttgcccgtttcggtaagaagagagttttctggatctgcatcagtttgattgaaatggcttacggtttcgctttgttgatgggtgctacttctgctttcttatggtctaagattgtcactgttttgggtcacgctgttttggcatcattcgttttgtatagagctaagtctattgatttgagatccaagacttccattacctctttctacatgttgatctggaagctaatgtacatggaatacttcttgatgccattagttagatga

SEQ ID NO: 37 - DNA, 990 bp, synthetic construct
  source 1..990: mol_type = other DNA
atggactctactgctgcctccaagatcttggttattggtggtactggttacatgggtcgttttctggtcgaagcttctgccaaggccggtcatcctacctttgctttagtacgtcacagtactgttaccaacccagacaagtcctctattattcactctttcaacaccctaggcgtcaatctattgttgggtgacattaatgatcatcaatctttggtcaaggccatcaagcaagctgatgttgtcatctctaccgttaaccaccaatatatttctgaccaatacaagattattagcgctatcaaggaagcaggtaacatcaaacgtttcttcccatctgaattcggtaacgacgttgtcagaactcacggtgtcgacggtgctaaggctttgttcgatatcaaggctaaattcagaagaaccatcgaagctgaaggtatccctcacacttacgtcgttgccaacttcttgactcaacacttcttgccaactagatccagattattggctattgctgctccattagataaggttgtcatattgggtgatggtaacaccaaggccacattcaacactgaagaaggtgttgctaccttcactatcagagctgtcgacgacccaagaactttgaacaagatcttatacattagaccaccagctaacactttgtcctacaacgatttggtttccttgtgggaaaagaagaccggtaacaccttggaaagagtttatgttccagaagaacaagttttgagattgatccaagaatcttcttacccattgaacatggctttgtccatctgtcacgctgcttacgttaagggtgaccacaccaactacgaaattgaaccatccttcggtgttgaagccttggaattgtacccagatgtcaagttcaccactgtggatgactacttggaacaaaacagagactgtactccattctacttaaatcaattgattccaatcactaacggtaccaaattttga

SEQ ID NO: 38 - DNA, 1629 bp, synthetic construct
  source 1..1629: mol_type = other DNA
atggctttcacttactccaccaagaaattggctttgttgtctattatcaccatcttcatctcctctttcccagaaccatctacctctgttgacttggaatccggtttcctgcaatgtttttccagtggtttgggtaactctaattccactgcagaattggttttgaccaagaactcgtcctcttacgcctccttgttgcaatcctccatccgtaacctaagattcatgggtacttccgtcccaaagccaactttgattgtcactccaagagacttgttccatatccaaacctccatcaaatgcagcgttaagcaaggtttgcaaattcgtgtcagatccggtggtcatgactacgaaggtttatcctacgtctccaacgatgacgttccattcttgattatcgacttgaccaacttgagatccatcactatcgatatcaaggatgaaactgcttgggttcaatctggtgctaccttgggtgaattatattacgctattgctaagaagtctaacgttcacggtttcccagctggttcttgtccaactgtcggtgttggtggtcacttctctggtggtggtttcggtaccattttcagaaagtacggtttagccgctgacaacgtcattgacgctcaaatggttgatgttaacggtaagatcttgaagaacagaactttgatgggtgaagatttgttttgggctatcagaggtggtggtggttcttccttcggtgttgttaccgcttggaaggtcaagttggtccatgttcctccaaaagttactgttttcaacattccaaagaccttggaccaaaatgcttctactttattccacaagtggcaaattgttgctgataagttgccaggtgaattgtttttgcactctgtcatgggtgtctctaacgctaactctatttcttcttcctcaggtggcggtagaaccgtcctagtctctttcaccggtctatacttgggtaccgttgaaaacttgttgccattgatgcaaaacaactttgctgaattaggtttgcaacacaacaactgtactgaaatgtcttggattcaatctgtacttttctttgccggctacgacccaatcaacgacagtttggaggtcttacttcaaagaaaccaaactttcggttctttcaaggccaaatctgactacgttatggaacctatcccaacctctggtttggaaggtttatggaacatgttgttggaagaaaattcttctccaactttgattttgactccatacggtggtagaatgtccgaaatctctgaatctgaaaccccattccctcacagaaacggatccatctacggtatccaatacttggtttactgggattcttctgaagaaacaccaaagcacatcgcctggatgagaagagtctactcttacatggtcttgtacgtttccaagttcccaagagctgcttacttgaactacagagatttggatattggtgtcaaccgtggtaacacttcttatgactatgaagaagctaagtcctggggtctcaaatacttcaagtgtaacttcgaaagattggccaaggttaaggctgaagttgacccatccaacttcttcagacacgaacaatccattccattgttgttttga

SEQ ID NO: 39 - DNA, 1446 bp, synthetic construct
  source 1..1446: mol_type = other DNA
atggaacacttttatatgtctttattgttactattcgttacattggtttctttgagcttattcttcttgatcttttaccacaacaagcacaacatgaacaacaacaacaatttgcctccaggtaagatgggttacccagttatcggtgaatctttggaatttttgtccatgggttggaagggtcaccctgaaaagttcattttcgacagaatggttagatactccagtgagttaattaagacttccatcttgggtgttccaactgtcatcttctgtggtccagcttgtaacaaattcttgttctctaacgaaaataagttggtgactgcttggtggccagactctgtcaacaaaatcttcccaaccacttctaattctaaggaagaatccaagaagatgagaaagttgttgccacaattcttgaagccagaagctttgcaacgttacgttggcattatggacactttagctcaaagacacttcgcgtctttgtgggaagaaaagacccatgtcaccgtttatcctttggctaagagatacactttcatgttggcctgtagattgttcatgtctgttgaagatgaaaaccacgttgccaagttcagagaaccattccatttgttggcctccggtatcatctctgtcccaattgacttgccatggactccattcaacagaggtatcaaggcttcaaacttcatcagaaaagaattgttgaaaattatcagacaaagaaaggtcgacttagctcaaggtgtcgcttccccaacccaagatatcttgtctcacatgttgctcacctgtgatgacgaaaacggtgaattcatgactgaattgaacatcgctgacaagattttgggtttgttgattggtggtcatgacaccgcttccgctgcttgcactttcattgtcaagtacctagctgaacttcctcacatttacgatagagtctaccaagaacaaatggaaattgctaactctaagtctccaggtgaattgctcaactgggatgatatcaacaagatgcgttactcttggaacgttgcttctgaagttatgagagttgccccaccattgcaaggtggtttccgtgaagctattaacgatttcgtctttaatggtttctccataccaaagggttggaagttgtactggtctgccaactccacccacaagaacccagaatacttcccagctccagaaaagtttgacccaaccagattcgaaggtaacggtccagccgcttacaccttcgtcccgttcggtggtggaccaagaatgtgtccaggtaaggaatacgctagattggaaatcctagttttcatgcacaacttggtcaaacgttttaagtgggaaatgttgattccagaagaaaagattgttgtcgatccattgcctatgccagccaacgacctaccaatcagattatacccacacaacacttga